(19) United States
(12) Patent Application Publication
(10) Pub. No.: US 2024/0318268 A1
(43) Pub. Date: Sep. 26, 2024

(54) METHOD AND KIT FOR DETECTING A REPLICATING RESPIRATORY VIRUS

(71) Applicants: BIOMÉRIEUX, MARCY L'ETOILE (FR); HOSPICES CIVILS DE LYON, Lyon (FR); UNIVERSITE CLAUDE BERNARD LYON I, Villeurbanne (FR)

(72) Inventors: Sophie ASSANT, REYRIEUX (FR); Karen BRENGEL-PESCE, LES ABRETS EN DAUPHINE (FR); Marine MOMMERT, CASTRES (FR)

(73) Assignees: BIOMÉRIEUX, MARCY L'ETOILE (FR); HOSPICES CIVILS DE LYON, Lyon (FR); UNIVERSITE CLAUDE BERNARD LYON I, Villeurbanne (FR)

(21) Appl. No.: 18/572,835

(22) PCT Filed: Jul. 7, 2022
(86) PCT No.: PCT/FR2022/051366

§ 371 Date: Dec. 22, 2023

(30) Foreign Application Priority Data
Jul. 8, 2021 (FR) FR2107421

(51) Int. Cl. C12Q 1/70; A61B 10/00; C12Q 1/6851

(52) U.S. Cl. CPC C12Q 1/701; C12Q 1/6851; A61B 10/0051; C12Q 2600/158

(57) ABSTRACT

A method for determining, in vitro or ex vivo, the presence in a patient of an infection by a replicating respiratory virus, including a step i) of determining, in a test sample taken from the mouth or nose of said patient, the level of transcripts of at least one marker gene selected from the interferon-stimulated genes, referred to as ISGs; and associated kits.

### TECHNICAL FIELD

The present invention relates to the technical field of diagnostic methods and kits. In particular, a subject of the invention concerns methods and kits for determining whether or not a subject is infected with a replicating respiratory virus.

### PRIOR ART

Many techniques are available today for detecting the DNA or RNA of a virus in a subject. This is notably the case for the pathogenic SARS-COV-2 virus, where several PCR tests are available, predominantly performed on nasopharyngeal or salivary samples from the subjects concerned. Mention may notably be made of qRT-PCR tests, notably the SARS-COV-2 RESPI R-GENE® test from bioMérieux, the BioFire® COVID-19 test from bioMerieux, the SARS-CoV-2 ddPCR test from Bio-Rad, the RealTime SARS-COV-2 test from Abbott, the RealStar® RSV RT-PCR test from Altona-diagnostics, the ALINITY m RESP-4-PLEX ASSAY test from Abbott and the Cobas® SARS-COV-2 test from Roche Diagnostics, notably.

However, PCR results are often difficult to interpret in routine clinical practice and in the absence of clinical manifestations (Han, M. S. et al., 2021). In particular, it is possible for a PCR test to detect only fragments of the virus' DNA or RNA genome, and therefore the presence of a virus that is not active, i.e. that does not have the capacity to replicate in the subject's body. The presence of symptoms, notably those that may be described as mild (or weak), which may be associated with many viral or bacterial infections, is not a sign of the presence of a replicating respiratory virus either. Furthermore, during the autumn and winter months, when many respiratory viruses are circulating, it is sometimes difficult to associate symptoms such as fever, suspecting an infection, with the presence of a replicating respiratory virus which is directly responsible. There is therefore considerable interest in identifying a means for determining the presence in a subject of an infection with a replicating respiratory virus such as SARS-COV-2. This would make it possible, notably, to rapidly identify patients at risk of viral transmission, and alternatively to avoid quarantine measures for patients who are not, or who are no longer, a possible source of contamination, notably in the case of SARS-COV-2.

Although a respiratory viral infection, notably with SARS-COV-2, initially occurs via the upper respiratory tract, type I and III interferon (IFN-I and III)-mediated immunity in the nasal mucosa remains poorly characterized during a respiratory viral infection.

Recently, Monel B. et al., 2021 looked at the production of various interferons and interleukins in nasopharyngeal samples and studied the correlation between their production and the presence of infection with a replicating SARS-COV-2 virus. Other authors also studied the expression level of interferon-stimulated genes, known as ISGs, in patients with positive SARS-COV-2 PCR tests, confirming the role of interferons in the anti-SARS-COV-2 mucosal immune response (Mick, E. et al., 2020, and Ng, D. L. et al., 2021). However, in studies related to the level of ISG expression, the replicative or non-replicative nature of the virus corresponding to positive PCR tests has not been studied at all; nor has there been any mention of the probability of investigating the level of expression of interferon-stimulated genes, known as ISGs, in order to assess contagiousness.

The publication by Gupta R. K. et al., 2021, relates to the evaluation of the diagnostic accuracy of transcriptomic signatures of viral infection in order to predict the positivity of a nasopharyngeal PCR test for SARS-COV-2. According to the results, several signatures reflecting the interferon-1 signalling pathway make it possible to make a discrimination between a negative control test and a positive PCR test for SARS-COV-2. However, here again, the replicative nature of the virus was not assessed in the positive PCR tests, thus making it impossible to identify the contagious nature of the virus.

Patent application WO 2019/236768 also proposes a method for detecting a respiratory infection in a subject, based on the level of expression of one or more genes of the subject (host gene). A very long list of host genes is given, including some ISGs, such as IFIT1, IFIT2, IFIT3 and RSAD2, however these are not presented as being particularly advantageous to select. Furthermore, knowing whether or not the subject is infected with a replicating respiratory virus is not at all addressed in said patent application.

### DISCLOSURE OF THE INVENTION

In this context, the present invention relates to a method for determining, in vitro or ex vivo, the presence in a subject of an infection with a replicating respiratory virus, comprising a step i) of determining, in a test sample from the mouth or nose of said subject, the transcript level of at least one marker gene selected from among the interferon-stimulated genes, known as ISGs (hereinafter referred to as ISG marker gene, or more simply as marker gene or ISG, for the sake of simplicity).

Advantageously, in step i), the transcript level of one or more marker genes chosen from: IFI27, IFI44L, IFIT1, RSAD2, ISG15 and SIGLEC1, is determined. The transcript level of all these marker genes may be determined. In particular, the transcript level of one or more marker genes chosen from: IFI27, IFI44L, IFIT1 and RSAD2 is determined. Advantageously, the transcript level of all the marker genes IFI27, IFI44L, IFIT1 and RSAD2 is determined. In cases where the transcript level of several marker genes is determined, the transcript level for each marker gene may be determined independently, or the different transcripts of all the marker genes used may be determined simultaneously, the level of which is determined globally. When the transcript level of several marker genes is determined independently, data (notably a score as used in the examples) representing the transcript level of all these marker genes may be calculated.

Advantageously, the test sample is an oropharyngeal or nasopharyngeal sample or a saliva sample.

In the context of the invention, also advantageously, step i) consists in determining the mRNA level of said marker gene(s).

The methods according to the invention, whatever their embodiment, may be performed on a sample from a subject showing symptoms of an infection, or on a sample from a subject who is asymptomatic. The method according to the invention is particularly advantageous in the case of asymptomatic subjects or subjects presenting symptoms that can be described as mild, such as fatigue, aches and pains, muscle pains, fever, respiratory symptoms, notably a cough without pneumopathy, headache, sore throat, malaise, nausea, vomiting, diarrhoea, anosmia or ageusia.

In the context of the invention, the test sample may be obtained or taken at any time. In the case of symptomatic subjects, the test sample may be obtained or taken before or after the onset of symptoms. The test sample may come from any type of subject, from a subject who has or has not been diagnosed as infected with a respiratory virus, from a subject who is awaiting a screening or diagnostic result for a respiratory virus, notably by detecting DNA or RNA of said virus, etc.

According to certain embodiments, the methods according to the invention, regardless of the embodiment variant thereof, may be performed on a sample from a subject who does not have neutralizing autoantibodies directed toward interferon α and/or neutralizing autoantibodies directed toward interferon ω.

In particular, the method according to the invention may comprise the following steps:
i) determining the transcript level of at least one marker gene chosen from the ISGs, for said test sample of the subject,
ii) comparing the transcript level of said at least one marker gene obtained, with a reference level.

The methods according to the invention include a step iii) which is that of concluding whether or not said subject is infected with a replicating respiratory virus. Such a conclusion is reached using at least one threshold value. The term “threshold value” means a predetermined value which is relevant for reaching a conclusion as to the presence or absence of infection with a replicating respiratory virus in a subject. A comparison is made between a threshold value and the transcript level of at least one ISG marker gene determined for the test sample of said subject, or with data (notably a score) obtained from the transcript level of at least one ISG marker gene determined for the test sample of said subject. Said data (or score) may notably be obtained from the transcript levels of several ISG marker genes determined for the test sample of said subject.

Depending on the result of the comparison, notably if the transcript level of at least one ISG marker gene determined for the test sample of said subject, or the data or score obtained from the transcript level of at least one ISG marker gene determined for the test sample of said subject, is different, notably greater than the threshold value, it is concluded that said subject is infected with a replicating respiratory virus.

According to a first embodiment of the methods according to the invention, in step iii), it may be concluded whether or not said subject is infected with a replicating respiratory virus, on the basis of the result of step ii), i.e. of the comparison between the transcript level of said at least one marker gene obtained, and the reference level of said marker gene, which then acts as a threshold value. This is particularly the case when, in the method according to the invention, the transcript level of a single ISG marker gene is determined. This is also the case when the transcript level of several ISG marker genes is determined globally and this global level is compared with a global reference level.

In this first embodiment, generally speaking, the reference level used as a threshold value, with which the transcript level of a marker gene is compared, corresponds to the transcript level of said marker gene, in a reference subject, and in particular in a subject not infected with a replicating respiratory virus, or in a population of such subjects. Subjects not infected with a replicating respiratory virus include subjects who are positive in a test for detecting the presence of a respiratory virus, but for whom the virus is not replicative, and subjects not infected with a respiratory virus (negative in a test for detecting the presence of a respiratory virus). In particular, the reference level corresponds to the transcript level of said marker gene, in a subject showing no infection with a replicating respiratory virus, whether positive or not to a test for detecting a respiratory virus, and who, preferably, does not have neutralizing autoantibodies directed toward interferon α and/or neutralizing autoantibodies directed toward interferon ω, or the reference level corresponds to the transcript level of said marker gene in a population of such subjects showing no infection with a replicating respiratory virus, whether or not they are positive in a test for detecting a respiratory virus, and who preferably do not have neutralizing autoantibodies directed toward interferon α and/or neutralizing autoantibodies directed toward interferon ω.

According to a first variant of the first embodiment, the transcript level of a single marker gene chosen from the ISGs may be determined in step i) and it is concluded that said subject is infected with a replicating respiratory virus if there is a difference with the reference level of said marker gene. Thus, the invention relates to a method for determining in vitro or ex vivo the presence in a subject of an infection with a replicating respiratory virus, comprising the following steps:
i) determining the transcript level of a single marker gene selected from the ISGs, in a test sample from the mouth or nose of said subject,
ii) comparing the transcript level of said marker gene obtained, with a reference level, used as a threshold value, said reference level being the transcript level of said marker gene, in a reference subject, and in particular in a subject not infected with a replicating respiratory virus, who preferably does not have neutralizing autoantibodies directed toward interferon α and/or neutralizing autoantibodies directed toward interferon ω, or in a population of such subjects, and
iii) concluding that an infection with a replicating respiratory virus is present in said subject in the event of a difference with said reference level of said marker gene.

In particular, said difference corresponds to a transcript level of said marker gene for the test sample which is greater than that of the reference level.

In such cases, said marker gene selected from the ISGs is preferably IFIT1 or IFI44L.

According to a second embodiment variant of the first embodiment:
the transcript levels of several marker genes selected from the ISGs may be determined in step i), for the test sample, and
the transcript level of each marker gene determined for the test sample may be compared with the reference level of said corresponding marker gene.

In this second embodiment variant, it may be concluded that said subject is infected with a replicating respiratory virus if there is a difference with the corresponding reference level, used as a threshold value, for at least one of the marker genes, for which the transcript level has been determined in the test sample.

Thus, the invention relates to a method for the in vitro or ex vivo determination of the presence in a subject of an infection with a replicating respiratory virus, comprising the following steps:
i) determining the transcript levels of several marker genes selected from the ISGs for the test sample, in a test sample from the mouth or nose of said subject,
ii) comparing the transcript level of each marker gene determined in the test sample with a reference level corresponding to said marker gene, used as a threshold value, for each marker gene considered, said reference level being the transcript level of said marker gene, in a reference subject, and in particular in a subject not infected with a replicating respiratory virus, who preferably does not have neutralizing autoantibodies directed toward interferon α and/or neutralizing autoantibodies directed toward interferon ω, or in a population of such subjects, and
iii) concluding that said subject is infected with a replicating respiratory virus, in the event of a difference with the corresponding reference level, for at least one of the marker genes, for which the transcript level has been determined in the test sample.

In particular, said difference corresponds to a transcript level of said marker gene for the test sample which is greater than that corresponding to the reference level of said marker gene.

Advantageously, in the second embodiment variant of the first embodiment, in step i), the transcript level of one or more marker genes chosen from: IFI27, IFI44L, IFIT1, RSAD2, ISG15, and SIGLEC1, is determined. The transcript level of all these marker genes may be determined. In particular, the transcript level of one or more marker genes chosen from: IFI27, IFI44L, IFIT1 and RSAD2 is determined. Advantageously, the transcript level of all the marker genes IFI27, IFI44L, IFIT1 and RSAD2 is determined.

According to a second embodiment of the methods according to the invention, in step iii), it may be concluded whether or not said subject is infected with a replicating respiratory virus, by performing an intermediate step iibis) leading to data obtained from the transcript level of at least one marker gene selected from the ISGs, for the test sample. This is particularly the case when the transcript levels of several ISG marker genes are determined in step i), for the test sample. A comparison step ii) may be performed during which the transcript level of each ISG marker gene is compared to a reference level of said marker gene, said comparison notably taking the form of calculating a ratio, notably the ratio between said transcript level of each marker gene and a reference level of said marker gene. An intermediate step iibis) may then be carried out: this step may consist in calculating a score representing the results of the comparisons performed for each marker gene. Such a score is, for example, the median of all the ratios obtained (in particular, transcript level of each ISG marker gene for the test sample/reference level corresponding to said marker gene). The conclusion may then be reached by comparing this score with a threshold value, notably 1.5, to conclude whether or not there is infection with a replicating respiratory virus in the subject of interest. If the score obtained for the test sample is greater than this threshold value, notably 1.5, it is concluded that there is infection with a replicating respiratory virus.

Advantageously, in the second embodiment of the methods according to the invention, in step i), the transcript level of one or more marker genes chosen from: IFI27, IFI44L, IFIT1, RSAD2, ISG15, and SIGLEC1, is determined. The transcript level of all these marker genes may be determined. In particular, the transcript level of one or more marker genes chosen from: IFI27, IFI44L, IFIT1 and RSAD2 is determined. Advantageously, the transcript level of all the marker genes IFI27, IFI44L, IFIT1 and RSAD2 is determined.

Thus, as an example of the second embodiment of the methods according to the invention, mention may be made of a method for determining in vitro or ex vivo the presence in a subject of an infection with a replicating respiratory virus, comprising the following steps:
i) determining the transcript levels of several marker genes selected from the ISGs, in a test sample from the mouth or nose of said subject,
ii) comparing the transcript level of each marker gene determined in the test sample with a reference level corresponding to said marker gene, calculating the ratio between said transcript level and said reference level of said marker gene, iibis) calculating the median of all the ratios obtained in step ii), and
iii) concluding that said subject is infected with a replicating respiratory virus when said median calculated in step iibis) is greater than a threshold value, which is preferably equal to 1.5.

In such a method, the value of 1.5 may thus be considered as the threshold value used to conclude whether or not there is infection with a replicating respiratory virus.

Advantageously, in the second embodiment of the methods according to the invention, for each marker gene for which the transcript level in the test sample is determined in step i), the reference level used for calculating the ratio is the transcript level of said marker gene, in a subject not infected with a respiratory virus, or in a population of such subjects of said marker gene. A subject not infected with a respiratory virus is, in particular, a subject who is negative to a test for detecting the presence of a respiratory virus. Preferably, the reference level used for calculating the ratio is the transcript level of said marker gene, in a subject not infected with a respiratory virus, not having neutralizing autoantibodies directed toward interferon α and/or neutralizing autoantibodies directed toward interferon ω or in a population of such subjects.

The methods according to the invention, whatever their embodiment, may also include a step performed prior to or concomitantly with step i), during which the test sample is subjected to a diagnostic test, by detection of DNA or RNA, notably by PCR or RT-PCR, of a respiratory virus, and notably of a SARS-COV-2 virus. In particular, said diagnostic test is performed prior to step i) and gives a positive result.

According to particular embodiments, in the methods according to the invention, the test sample is subjected to a diagnostic test, by detection of DNA or RNA, notably by PCR or RT-PCR, of a respiratory virus chosen from the SARS-COV-2 virus and its variants and influenza viruses, notably influenza A, B and C, said diagnostic test having given a positive result. Such a diagnostic test may also be performed prior to or concomitantly with step i), and the positive or negative result may be obtained before or after determining the presence or absence in said subject of an infection with a replicating respiratory virus.

In the context of the invention, whatever the embodiment variant, the transcript level of the marker gene(s) is determined, in particular at the mRNA level. The transcript level is notably determined by hybridization, amplification or sequencing, and in particular by RT-qPCR.

According to the invention, the transcript level of the marker gene(s) obtained may be a normalized value relative to the transcript level of one or more housekeeping genes, notably chosen from DECR1, HPRT1 and PPIB.

The present invention also relates to a kit for determining in vitro or ex vivo the presence in a subject of an infection with a replicating respiratory virus, comprising:—at least one means for determining the transcript level of a marker gene selected from the ISGs, notably chosen from oligonucleotides,
at least one threshold value stored on a computer-readable medium and/or used in the form of a computer-executable code configured to compare the transcript level of said marker gene, determined with the determination means or data obtained from said transcript level of the marker gene, with said threshold value.

If several ISG marker genes are used, the kit will generally include at least one means for determining the transcript level for each of them.

If several ISG marker genes are used, the kit may include a threshold value and/or reference level for each of them, stored on a computer-readable medium and/or used in the form of a computer-executable code.

If several marker genes are used, the kit may also include an executable code for generating global reference data from the comparison of each marker gene with its reference data and for comparing this global data with a threshold value. In this case, the comparison of the level of each marker gene with its reference level may take the form of calculating a ratio, as previously explained.

According to certain embodiments, said threshold value corresponds to the transcript level of said marker gene in a subject who has no infection with a replicating respiratory virus and who, preferably, has no neutralizing autoantibodies directed toward interferon α and/or has no neutralizing autoantibodies directed toward interferon ω, or in a population of subjects with no infection with a replicating respiratory virus and who preferably do not have neutralizing autoantibodies directed toward interferon α and/or do not have neutralizing autoantibodies directed toward interferon ω.

According to certain embodiments, such a kit according to the invention also comprises at least one reference level for said marker gene, stored on a computer-readable medium and/or used in the form of computer-executable code configured to compare the transcript level of the marker gene determined with the determination means with said reference level, said reference level preferably being the transcript level of said marker gene, in a subject not infected with a respiratory virus, or in a population of such subjects.

Advantageously, such kits according to the invention also comprise at least one means for detecting a respiratory virus, and notably at least one means for detecting a SARS-COV-2 virus and/or at least one means for detecting the influenza virus (influenza A, B or C).

The present invention also relates to a kit for determining in vitro or ex vivo the presence in a subject of an infection with a replicating respiratory virus, comprising:
at least one means for determining the transcript level of a marker gene selected from ISGs, notably chosen from oligonucleotides,
at least one means for detecting a respiratory virus, and notably at least one means for detecting a SARS-COV-2 virus.

Advantageously, such a kit also comprises at least one threshold value stored on a computer-readable medium and/or used in the form of a computer-executable code configured to compare the transcript level of said marker gene determined with the determination means, or data obtained from said transcript level of the marker gene, with said threshold value.

According to certain embodiments, said threshold value corresponds to the transcript level of said marker gene in a subject who has no infection with a replicating respiratory virus and who, preferably, has no neutralizing autoantibodies directed toward interferon α and/or has no neutralizing autoantibodies directed toward interferon ω, or in a population of subjects with no infection with a replicating respiratory virus and who preferably do not have neutralizing autoantibodies directed toward interferon α and/or do not have neutralizing autoantibodies directed toward interferon ω.

According to other embodiments, such a kit according to the invention also comprises at least one reference level for said marker gene, stored on a computer-readable medium and/or used in the form of computer-executable code configured to compare the transcript level of the marker gene determined with the determination means with said reference level, said reference level preferably being the transcript level of said marker gene, in a subject not infected with a respiratory virus, or in a population of such subjects.

Whatever their embodiment, the detection kits according to the invention may also comprise a negative control sample, such as a sample making it possible to ensure the absence of contamination, and/or a positive control sample which corresponds to the transcript level of said marker gene at a concentration representative of the transcript level in a subject with infection with a replicating respiratory virus or in a population of subjects with infection with a replicating respiratory virus.

The invention will be better understood in the light of the detailed description below, with reference to the drawings.

### BRIEF DESCRIPTION OF THE DRAWINGS

FIG. 1 shows the correlation between the nasal type I/III ISG score obtained from a nasopharyngeal swab (NP) and the blood type I/III ISG score obtained from a PAXgene™ whole blood sample (PX, n=75 longitudinal samples from 23 patients). The different scores are measured using FilmArray® technology. Spearman's correlation coefficient is shown.

FIG. 2 shows that the nasal (NP for nasopharyngeal) ISG score determined by FilmArray® technology correlates with IFN-α2 levels (fg/ml) in plasma (n=39 samples with detectable values from 23 patients), measured by a single molecule array (SIMOA®) using a commercially available kit. Spearman's correlation coefficient is shown.

FIG. 3 shows kinetic measurements of NP (gray square) and blood (black square) ISG scores after diagnosis of COVID-19. n=75 longitudinal samples from 23 patients. Loess fit curves represent local polynomial regressions as determined by the Loess method. The 95% CI is indicated (gray area).

FIG. 4 represents the scatter plot of the NP ISG score in patients with negative or positive nasal SARS-COV-2 viral culture (n=30 longitudinal samples from 12 patients). The black circles represent the negative viral culture samples and the empty circles the positive viral culture sample, the triangles correspond to the positive cell culture samples with no cytopathic effect.

FIG. 5 is a Receiver Operating Curve (ROC) distinguishing between positive and negative SARS-COV-2 viral culture results. The area under the curve and the Youden index are shown and represent the ability of the NP type I/III ISG score to separate positive and negative viral cultures.

FIG. 6 represents the nasal type I/III ISG score measured after 1/10 serial dilutions (10⁰ to 10⁻²) of NP samples (solid line, FilmArray®) and also SARS-COV-2 detection (Ct detection, dashed line, SARS-COV-2 R-gene® kit). The lower gray area indicates the threshold value of the normal nasal type I/III ISG score (1.99) and the upper gray area indicates the value of 6.75 of the type I/III nasal ISG score associated with a viral culture of a replicating virus.

FIG. 7 compares the normalized nasal viral load and the NP type I/III ISG score in severely affected COVID-19 patients without anti-IFN autoantibodies (nAb) (empty circles) or with neutralizing anti-IFN-α autoantibodies (black triangles). The gray area represents the viral load obtained in 90% of mild COVID-19 patients with a nasal type I/III ISG score >6.75 (separation value associated with viral culture of a replicating virus).

FIG. 8 is a ROC curve discriminating between the positive and negative viral PCR result for type I/III IFN score. The area under the curve, Youden index and cut-off point are shown and represent the ability of the nasal type I/III IFN score to discriminate between PCR positive and negative samples.

FIG. 9 is a graph representing the IFN I/III score as a function of viral PCR, NP samples from individuals with negative viral PCR (n=23) or positive PCR for influenza virus (influenza B) (n=42). Comparisons were performed using a Kruskal-Wallis test followed by a Dunn multiple comparison test (p<0.0001 ****).

FIG. 10 shows graphs representing (A) IFI27 mRNA expression level versus viral PCR, (B) IFI44L mRNA expression level versus viral PCR, (C) RSAD2 mRNA expression level versus viral PCR and (D) IFIT1 mRNA expression level versus viral PCR. The mRNA expression level is expressed as the log 2 ratio of the ISG RQ to the median RQ of the same ISG from an HV population. The RQs were calculated for the NP NEG (n=23) and GRB (n=42) samples by converting the Ct obtained with the BioFire® IFN FilmArray® pouch panel into GQ (2-CT) divided by the geometric mean of the GQs of the housekeeping genes. NP (nasopharyngeal), GRB (influenza B virus), HV (healthy volunteers), RQ (relative quantity) GQ (gross quantity).

FIG. 11 is a graph representing the FilmArray IFN I/III score as a function of viral culture, of NP samples from patients infected with influenza virus (influenza B) (n=7; n=28) and RSV (n=25; n=7). Comparisons were performed using the non-parametric Wilcoxon Mann and Withney test (p<0.0001 ***).

FIG. 12 is an ROC curve discriminating between positive and negative influenza viral culture result for nasal IFN I/III score. The area under the curve, Youden index and cut-off point are shown and represent the ability of the nasal IFN I/III score and viral load to discriminate between positive and negative cultures.

### DETAILED DESCRIPTION OF THE INVENTION

Certain terms and expressions used in the context of the invention are detailed hereinbelow.

The term “subject” denotes a mammal and preferably a human being. Said subject, who is a human being, may be a patient who has come into contact with a health professional, such as a doctor (for instance, a general practitioner) or a medical structure or health establishment (for instance, a hospital or clinic, and more particularly an emergency department, a resuscitation department, an intensive care unit or a continuous care unit, or a medicalized structure for the elderly, of the nursing home type).

As a “sample from the mouth or nose of a subject”, it may be a sample taken from the subject's nose or mouth, or it may be a sample of air released by the subject. The sample may be a nasal sample or a saliva sample, but is preferably an oropharyngeal or nasopharyngeal sample. The oropharyngeal or nasopharyngeal samples are notably obtained by performing a swab sampling, respectively at the back of the mouth or nose of a subject.

The term “replicating virus” means a virus which is capable of multiplying in said subject. Thus, a subject infected with a replicating virus is a transmission vector for such a virus to another subject. In particular, the notion of a replicating virus excludes virus fragments which have lost their ability to reproduce. Such virus fragments will give a positive result in a DNA or RNA virus detection test, but they may not have the capacity to replicate in the subject, who therefore cannot be a transmission vector for the corresponding virus. In the presence of a replicating virus, the risk of transmitting the virus is significant, which is not the case in the absence of the ability to replicate. The ability of a virus to multiply (replicate) and therefore its replicative nature may be defined by the fact that it multiplies (increase in viral load), which may be reflected by the observation of a cytopathic effect when it is cultured, or when part of the test sample taken which is likely to contain such a replicating virus is cultured, on epithelial cells, such as Vero cells. Vero cells that are particularly suitable for testing the replication capacity of a virus are notably those deposited under ATCC number CCL-81®, or Vero E6 cells deposited under ATCC number CRL-1586®. A cytopathic effect may notably be materialized by the presence of patches of lysis, which is characteristic of the presence of replicating viral particles. Protocols for culturing viruses or samples on epithelial cells, which may be used to demonstrate viral multiplication, are widely known to a person skilled in the art and are described, for example, in Leland D. S. et al., 2007, and more specifically in Stelzer-Braid S., et al., 2020. However, such methods require a certain amount of time, notably at least 96 hours, whereas with the methods according to the invention, it is possible to obtain a conclusion as to the presence or absence of a replicating respiratory virus in a time of typically 45 minutes to 6 hours. Given that a subject infected with a replicating virus is a transmission vector for such a virus to another subject, it is particularly advantageous in the context of the invention to propose methods, uses and kits for determining whether or not a subject is infected with a replicating respiratory virus, notably in the context of the SARS-CoV-2 virus pandemic.

The term “respiratory virus” means a virus that infects the respiratory tract and/or lungs. Such viruses are classically found in samples taken from a subject's nose, throat and/or mouth, notably in nasal or nasopharyngeal samples (which require sampling deeper in the nose), oropharyngeal samples (which require sampling from the back of the throat) or saliva. Examples of respiratory viruses that may be mentioned include seasonal coronaviruses, the SARS-COV-2 virus, whatever its variants, the influenza virus (influenza A, B and C), the respiratory syncytial virus (RSV), rhinoviruses, metapneumoviruses, parainfluenza viruses and adenoviruses, etc. To date, the known variants of the SARS-COV-2 virus are notably its so-called English, Brazilian, South African, American and Indian variants. Knowledge of these variants and their names is evolving (https://www.who.int/docs/default-source/coronaviruse/situation-reports/20210225_weekly_epi_update_voc-special-edition.pdf?sfvrsn=1eacfa47_7&download=true) and the invention is applicable to each of them. The “English variant” of SARS-COV-2 was discovered on Sep. 20, 2020, in Kent (south-east England) (https://www.lemonde.fr/blog/realitesbiomedicales/tag/vui-202101-01/). On December 14, the United Kingdom reported the circulation of this variant to the World Health Organization (WHO). Initially named VUI 202012/01 (Variant Under Investigation, year 2020, month 12, variant 01), it was quickly renamed VOC 202012/01 (Variant Of Concern) on Dec. 18, 2020. It is part of lineage B.1.1.7 in the phylogenetic tree and includes deletion 69-70, also denoted ΔH69/V70. The Brazilian variant P.1 is a descendant of lineage B.1.1.28 (which circulated extensively in the state of Rio de Janeiro and probably emerged in Brazil in February 2020). This Brazilian P.1 variant contains numerous mutations, in particular E484K, K417T and N501Y. The Japanese variant derived from a lineage present in Brazil (B.1.1.28) contains a very high number of genetic changes. It includes twelve amino acid mutations in the spike protein, notably the mutations N501Y, E484K and K417T. The South African variant named 501Y.V2 (lineage B.1.351) also contains a number of mutations, including three, K417N, E484K and N501Y, located in the RBD domain of the spike protein, the receptor-binding domain.

According to a particular embodiment, the respiratory virus is chosen from SARS-COV-2 viruses, influenza viruses (influenza A, B and C), respiratory syncytial virus (RSV), rhinoviruses, metapneumoviruses, parainfluenza viruses and adenoviruses, and preferably from SARS-COV-2 viruses and its variants, and influenza viruses (influenza A, B and C).

Thus, according to one variant of this embodiment, the respiratory virus is chosen from the SARS-COV-2 virus and its variants. According to another variant of this embodiment, the respiratory virus is chosen from influenza A, B and C viruses, preferably influenza B virus.

Interferons (IFNs) are among the first molecules produced in the body following recognition of a pathogen by innate immune receptors. Interferons are widely described in the literature. Reference may be made, for example, to Manry J. et al., 2012 and Hertzorg P. J. et al., 2016. Human IFNs have been classified into three types, based on the receptors they use, their homology and their chromosomal location. There are 17 type I IFNs: 13 subtypes of IFN-α, and IFN-β, ε, κ and ω. The genes encoding these IFNs are located on chromosome 9 and bind to a single receptor composed of the IFNAR1 and IFNAR2 subunits. IFN-γ, the only type II IFN, is the product of a gene located on chromosome 12, and uses a receptor composed of the IFN-γR1 and IFN-γR2 subunits. Type III IFNs have been described more recently and correspond to the cytokines IL-28A, IL-28B and IL-29 (also known as IFN-λ2, IFN-λ3 and IFN-λ1). The genes encoding these IFNs are located on chromosome 19. Interferons regulate the expression of several hundred genes, the so-called interferon-stimulated genes (or ISGs). Secreted type I IFNs bind to interferon receptors (IFNARs) which perform signalling via Janus kinase 1 (JAK1) and tyrosine kinase 2 (Tyk2) and activate the production of STAT1, STAT2 and IRF9. The latter form the ISGF-3 complex, which translocates to the nucleus to bind to interferon-stimulated response elements (ISREs) in the genome, thus inducing the expression of hundreds of ISGs, most of which have antiviral activity. Secreted type III IFNs bind to another receptor, consisting of IFNLR and IL10RB, and yet activate the same ISGF-3, inducing largely overlapping ISGs (Lazear, H. M. et al., 2019). IFN-I and III are involved as the first line of defence against infection, as they promote viral clearance, induce tissue repair, and stimulate the adaptive immune response.

Examples of ISG genes that may be mentioned include ADAR, BST2, CHUK, DDX58, EIF2AK2, FOS, GBP2, HLA-A, HLA-B, HLA-C, HLA-E, IFI35, IFI6, IFIH1, IFIT2, IFIT3, IFITM1, IFITM2, IFITM3, IFNA14/16, IFNA2, IFNA4/7/10/17/21, IFNA5, IFNA6, IFNA8, IFNAR1, IFNAR2, IFNB1, IKBKB, IKBKE, IKBKG, IRF1, IRF3, IRF4, IRF7, IRF9, JAK1, JUN, MAP3K7, MAPK14, MAPK8, MAVS, MX1, MYD88, NFKB1, OAS1, OAS2, OAS3, OASL, PSMB8, PTPN6, RIPK1, RNASEL, SAMHD1, SOCS1, SOCS3, STAT1, STAT2, TBK1, TLR7, TRAF3, TRAF6, TYK2, XAF1, IFNL1, IFNL2/3, IFNL4, IFNLR1, IL10RB, IFI27, IFI44L IFIT1, RSAD2, ISG15, and SIGLEC1. ISGs chosen from IFI27, IFI44L, IFIT1, RSAD2, ISG15 and SIGLEC1 (Picard C., et al., 2018) are preferred in the context of the invention. Their sequences are given in the ENSEMBL and NCBI databases. These ISGs are notably stimulated by type I and III interferons. IFI27, IFI44L, RSAD2 and IFIT1, which are four ISGs regulated via IFN-stimulated gene factor 3 (ISGF3)-dependent ISRE elements, are even more preferred in the context of the invention.

In particular, in the context of the invention, the transcript level of one or more ISG-type marker genes, chosen from: IFI27, IFI44L, IFIT1, RSAD2, ISG15, and SIGLEC1, is determined (Picard C, et al., 2018).

The term “neutralizing antibody” means an antibody which neutralises the effect of the target against which it is directed.

The term “autoantibodies” means self-generated antibodies directed toward self-proteins. Such autoantibodies may be generated in the absence of infection.

The term “symptom” means any type of symptom, for example, symptoms that may be described as mild, such as fatigue, aches and pains, fever, respiratory symptoms, such as cough without pneumopathy, headache, anosmia or ageusia, or severe symptoms, which require hospitalization in intensive care or respiratory assistance. In the case of SARS-COV-2, a classification of asymptomatic to critical SARS-COV-2 infections (for people who test positive for SARS-COV-2 using a PCR test or an antigenic test) has been established on the basis of symptoms (Trouillet-Assant, S. et al., 2020 and https://www.covid19treatmentguidelines.nih.gov/overview/clinical-spectrum/):
asymptomatic or presymptomatic infection: people who test positive for SARS-CoV-2 using a virological test, but who have no symptoms compatible with COVID-19;
Mild infection: people who have any of the various signs and symptoms of COVID-19 (for example, fever, cough, sore throat, malaise, headache, muscle aches, nausea, vomiting, diarrhoea, loss of taste and smell), but who do not have shortness of breath, dyspnoea or abnormal chest imaging;
moderate infection: people who show signs of lower respiratory tract disease on clinical assessment or imaging and who have an oxygen saturation (SpO2)≥94% on room air and at sea level;
severe infection: people who have an SpO2<94% on room air and at sea level, a ratio of arterial partial pressure of oxygen to the fraction of inspired oxygen (PaO2/FiO2)<300 mm Hg, a respiratory rate >30 breaths/min, or infiltrated lungs >50%;
critical infection (sepsis): people suffering from respiratory failure, septic shock and/or multiple organ dysfunction.

In particular, the methods according to the invention may be performed on samples from subjects with asymptomatic, presymptomatic, mild or moderate SARS-COV-2 infection.

A “comparison” or verification of a “difference” between two values or levels may be performed by any known technique, notably by any automated, computerized or computer-assisted technique. The comparison or verification of a “difference” may involve the calculation of a ratio or difference.

In the context of the invention, a conclusion as to whether or not the subject from whom the test sample was taken is infected with a replicating respiratory virus may also be performed by any automated, computer-operated or computer-assisted technique.

The “median” of a set of values is the value in the middle of the lowest and highest values in the set of values.

A test for detecting a respiratory virus, a test for diagnosing a respiratory virus or a test for detecting the presence of infection with a respiratory virus is taken to mean any test known to a person skilled in the art enabling such a conclusion to be drawn, notably tests for detecting the DNA or RNA of said respiratory virus, notably PCR tests or antigenic tests.

The invention proposes to use the transcript level of at least one marker gene selected from among the interferon-stimulated genes, known as ISGs, determined in a test sample from the mouth or nose of a subject, to determine whether or not said subject has an infection with a replicating respiratory virus.

To this end, the invention proposes methods for determining in vitro or ex vivo the presence in a subject of an infection with a replicating respiratory virus, comprising a step i) of determining, in a test sample from the mouth or nose of said subject, the transcript level of at least one marker gene selected from the interferon-stimulated genes, known as ISGs. In the methods according to the invention, the transcript level of at least one marker gene selected from the interferon-stimulated genes, known as ISGs, is used to determine whether or not said subject is suffering from an infection with a replicating respiratory virus. That is to say, in the methods according to the invention, a conclusion as to the possible presence of infection with a replicating respiratory virus in said subject is reached by taking into account the transcript level of an ISG marker gene, or even of several transcript levels of different ISG marker genes. The conclusion may be reached on this basis alone (i.e. from the transcript level of a single ISG marker gene or from several transcript levels of different ISG marker genes) or by taking into account genes other than ISGs, although this is not preferred in the context of the invention.

According to the invention, the transcript level is determined by quantitative detection of one or more transcripts of the gene, and in particular of the messenger RNA (mRNA) type, in the sample of interest (whether this is the test sample or a reference sample). Determining the transcript level of an ISG therefore involves a quantitative measurement representing the quantity of transcripts of said ISG, notably the quantity of mRNA of said ISG, in the test sample.

The term “transcript” means the RNAs, and in particular messenger RNAs (mRNAs), produced by gene transcription. More precisely, transcripts are the RNAs produced by the transcription of a gene followed by post-transcriptional modifications of the pre-RNA forms. In the context of the present invention, measurement of the transcript level of the same gene may include the level of identical or different transcripts. Preferably, in the context of the invention, the determination of the transcript level relates to the determination of the level of mRNA of said gene. In the case of an mRNA transcript, detection may be performed by a direct method, by any method known to a person skilled in the art making it possible to determine the presence of said transcript in the sample, or by indirect detection of the transcript after transformation of the latter into DNA, or after amplification of said transcript or after amplification of the DNA obtained after transformation of said transcript into DNA. Numerous methods exist for detecting nucleic acids (see for example Kricka et al., 1999, and Relier G. H. et al., 1993). Gene expression may notably be measured by Reverse Transcription-Polymerase Chain Reaction or RT-PCR, preferably by quantitative RT-PCR or RT-qPCR (for example using FilmArray® technology), by sequencing (including high-throughput sequencing) or by hybridization techniques (for example using hybridization microarrays or techniques such as NanoString® nCounter®). A technique without RNA or DNA extraction may be performed, as is the case with the FilmArray® technique (Poritz et al. 2011).

Determining the transcript level of a gene makes it possible to determine the quantity of one or more transcripts of said gene present in the sample of interest or to give a derived value which is representative of the quantity of transcripts present in the sample of interest. Such a derived value representing the quantity may, for example, be the absolute concentration, calculated using a calibration curve obtained from successive dilutions of a solution of amplicons of known concentration. It may also correspond to the normalized and/or calibrated value of the quantity of transcript, such as the CNRQ (Calibrated Normalized Relative Quantity) (Hellemans et al., 2007), which integrates the values of a reference sample (or of a calibrator) and one or more housekeeping genes (also called reference genes). Examples of housekeeping genes that may be mentioned include DECR1, HPRT1, PPIB, RPLP0, PPIA, GLYR1, RANBP3, 18S, GAPDH, ACTB, ABCF1, ALAS1, GUSB, HPRT1, MRPS7, NMT1, NRDE2, OAZ1, PGK1, SDHA, STK11IP and TBP.

In general, in the methods and uses according to the invention, whatever their embodiments, the transcript level of a selected marker gene determined for the test sample of the subject is compared with a reference level for said marker gene, which in certain embodiments is also used as a threshold value for drawing a conclusion as to the presence or absence of infection with a replicating respiratory virus in the subject of interest. Such a comparison may be performed in various ways, in a manner known to a person skilled in the art. In the context of the invention, it is also possible, for the comparison, to calculate a ratio between the transcript level of a selected marker gene and the reference level for said marker gene, notably before performing a comparison with the threshold value used.

Preferably, in the method according to the invention, whatever the embodiment, the transcript level of the selected marker gene(s) is normalized relative to the transcript level of one or more housekeeping genes (or reference genes), as is known to a person skilled in the art; even more preferably using one or more of the following housekeeping genes: DECR1 (chromosomal location of the gene according to GRCh38/hg38: chr8:90,001,352-90,053,633), HPRT1 (chromosomal location of the gene according to GRCh38/hg38: chrX:134,452,842-134,520,513) and PPIB (chromosomal location of the gene according to GRCh38/hg38: chr15:64, 155,812-64, 163,205). In such a case, the reference level used is also normalized beforehand, in the same way. Normalization, whether for the reference level or for the transcript level in the test sample, is performed before the comparison, notably before calculating a ratio between the transcript level in the test sample and the reference level. When a threshold value different from a reference level is used to reach a conclusion, this normalization may be taken into account when choosing the threshold value.

If the transcript level of a marker gene is normalized relative to the transcript level of one or more housekeeping genes, this of course implies that the method according to the invention includes determining the transcript level of the housekeeping gene(s) used for normalization.

According to a first embodiment variant, the transcript level of a single marker gene selected from the ISGs may be determined in step i) and it may be concluded that said subject is infected with a replicating respiratory virus, if comparison of the transcript level of said ISG marker gene with a predetermined threshold value shows that there is a difference with said given or predetermined threshold value corresponding to said marker gene. In particular, said difference corresponds to a transcript level for the test sample which is greater than that corresponding to the threshold value.

Advantageously, said threshold value corresponds to a reference level of said marker gene which is the transcript level of said marker gene in a subject who has no infection with a replicating respiratory virus and who, preferably, has no neutralizing autoantibodies directed toward interferon α and/or has no neutralizing autoantibodies directed toward interferon ω, or in a population of subjects with no infection with a replicating respiratory virus and who preferably do not have neutralizing autoantibodies directed toward interferon α and/or do not have neutralizing autoantibodies directed toward interferon ω.

Preferably, said difference between the transcript level of the selected ISG marker gene determined in the test sample and the reference level of said ISG marker gene (threshold value), corresponds to the fact that the transcript level of said ISG marker gene determined in the test sample is increased, or significantly increased, in particular by at least 10%, at least 20%, at least 30%, at least 40%, at least 50%, at least 60%, at least 70%, at least 80% or at least 90% relative to the reference level of said ISG. The increase considered relevant for reaching a conclusion will depend on the threshold value considered, and notably on the reference level used as the threshold value, and will be adapted accordingly by a person skilled in the art. In particular, if the threshold value corresponds to the reference level of said ISG marker gene which is the transcript level of said ISG marker gene in a subject showing no infection with a replicating respiratory virus and who, preferably, has no neutralizing autoantibodies directed toward interferon α and/or has no neutralizing autoantibodies directed toward interferon ω, or in a population of subjects with no infection with a replicating respiratory virus and who preferably do not have neutralizing autoantibodies directed toward interferon α and/or do not have neutralizing autoantibodies directed toward interferon ω, it may be sufficient for the transcript level of the selected ISG determined in the test sample to be simply greater than the threshold value. However, if a different reference level is taken as the threshold value, notably the level in subjects not suffering from a respiratory infection or, more generally, healthy subjects (with no infection whatsoever, as are blood donors in particular), the difference relative to the threshold value must be more significant, notably according to one of the above-mentioned % increases. An increase may be deemed significant after application of a statistical method well known to those skilled in the art, such as the Student or Wilcoxon test. Such a method is notably used in the examples.

In such cases of a method using a single marker gene to provide a conclusion, said marker gene selected from the ISGs may be any ISG gene given in the present description, but is preferably IFIT1 or IFI44L.

When several marker genes are used in the context of the invention, the comparison may be performed by comparing the transcript level of each selected marker gene with a given or predetermined threshold value for each marker gene considered. It is also possible to perform a more global comparison. In particular, it is possible to obtain the transcript level of several marker genes and to compare the overall transcript level of these marker genes with a single global threshold value. It is also possible to perform a comparison of the transcript level of each selected marker gene with a given threshold value for each marker gene. In such a case, advantageously, said threshold value corresponds to a reference level of said marker gene which is the transcript level of said marker gene in a subject showing no infection with a replicating respiratory virus and who, preferably, has no neutralizing autoantibodies directed toward interferon α and/or has no neutralizing autoantibodies directed toward interferon ω, or in a population of subjects showing no infection with a replicating respiratory virus and who preferably do not have neutralizing autoantibodies directed toward interferon α and/or do not have neutralizing autoantibodies directed toward interferon ω.

In this context, according to a second embodiment variant of the methods according to the invention:
the transcript levels of several marker genes selected from the ISGs may be determined in step i), for the test sample, and
the transcript level of each marker gene determined in the test sample is compared with the threshold value of said corresponding ISG marker gene.

In the second embodiment variant, it may be concluded that said subject is infected with a replicating respiratory virus if there is a difference with the threshold value for at least one of the marker genes, for which the transcript level has been determined in the test sample.

In particular, said difference corresponds to a level greater than that of the threshold value. Notably, it may be concluded that said subject is infected with a replicating respiratory virus if, for at least one of the ISG marker genes whose transcript level is selected, the transcript level of said ISG marker gene determined in the test sample is increased, or even significantly increased, in particular by at least 10%, at least 20%, at least 30%, at least 40%, at least 50%, at least 60%, at least 70%, at least 80% or at least 90% relative to the threshold value considered for said ISG. Here again, the increase considered relevant for reaching a conclusion will depend on the threshold value considered, and notably on the reference level used as the threshold value, and will be adapted accordingly by a person skilled in the art. In such cases, the methods according to the invention may involve determining the transcript level of 1 to 71 marker genes chosen from those given in the present description, and, preferably, the transcript level of 1 to 6 marker genes chosen by the ISGs, and notably chosen from IFI27, IFI44L, IFIT1, RSAD2, ISG15 and SIGLEC1. In particular, it may be concluded that said subject is infected with a replicating respiratory virus if, for at least one, or even at least two, or three to four, of the ISGs whose transcript level is selected, the transcript level of said ISG or ISGs determined in the test sample is increased relative to the corresponding threshold value, and notably according to a percentage increase defined previously.

In certain embodiments, a comparison may be performed by calculating a ratio of the transcript level of said selected marker gene determined for the test sample to a corresponding reference level for said marker gene. Alternatively, such a comparison may be performed by calculating the ratio of the reference level of said selected marker gene to the transcript level of said selected marker gene determined for the test sample. Then, an overall ratio corresponding to the median of said ratios may be calculated and used to provide a conclusion as to the presence or absence of infection with a replicating respiratory virus in said subject.

In a third embodiment variant, it may also be concluded that said subject is infected with a replicating respiratory virus, by performing the following steps:
for each marker gene for which the transcript level in the test sample is determined in step i), the ratio between said transcript level and the corresponding reference level of said marker gene is calculated,
the median of all the ratios obtained is then calculated and it is concluded that said subject is infected with a replicating respiratory virus if said median is greater than a threshold value, which may for example be equal to 1.5, and preferably greater than 2, 3, 4, 5 or 6. Such values are determined and adapted by a person skilled in the art, in a completely conventional manner, as a function of the subject and the reference level used to calculate the ratio, notably by applying a statistical method as previously described. In particular, the reference level of said marker gene for calculating the ratios may be the transcript level of said marker gene, in a subject not infected with a respiratory virus, or in a population of such subjects of said marker gene. A subject not infected with a respiratory virus is, in particular, a subject who is negative in a test for detecting the presence of a respiratory virus. Preferably, the reference level used for calculating the ratio is the transcript level of said marker gene, in a subject not infected with a respiratory virus, not having neutralizing autoantibodies directed toward interferon α and/or neutralizing autoantibodies directed toward interferon ω or in a population of such subjects.

In such cases, the methods according to the invention may include determining the transcript level for 1 to 71, and preferably for 1 to 6, marker genes chosen by the ISGs, and notably chosen from IFI27, IFI44L, IFIT1, RSAD2, ISG15 and SIGLEC1.

In particular, the method according to the invention comprises:
i) the determination of the transcript levels of several marker genes selected from IFI27, IFI44L, IFIT1, RSAD2, ISG15 and SIGLEC1, for the test sample,
ii) for each marker gene for which the transcript level in the test sample is determined in step i), the ratio between said transcript level and the reference level of said marker gene, is calculated,
iii) the median of all the ratios obtained is then calculated and it is concluded that said subject is infected with a replicating respiratory virus, when said median is greater than the threshold value which may, for example, be equal to 1.5, preferably greater than 2, 3, 4, 5 or 6.

In the context of this method, it is possible to determine in step i) and to use in steps ii) and iii), the transcript levels of all the marker genes IFI27, IFI44L, IFIT1 and RSAD2, or even of all the marker genes IFI27, IFI44L, IFIT1, RSAD2, ISG15 and SIGLEC1. According to a particular embodiment illustrated in the examples, only the transcript levels of the genes IFI27, IFI44L, IFIT1 and RSAD2 are determined and used as marker genes. In other words, the conclusion regarding the presence or absence in the subject of infection with a replicating respiratory virus does not take into account the transcript level of another marker gene. According to another particular embodiment, only the transcript levels of the genes IFI27, IFI44L, IFIT1, RSAD2, ISG15 and SIGLEC1 are determined and used as marker genes.

The threshold value or reference level are, in general, values determined upstream which are available when the method according to the invention is performed. The threshold value is a value which has previously been judged to be relevant for obtaining a conclusion as to the presence or absence of infection with a replicating respiratory virus. The reference level corresponds to the transcript level for the marker gene considered, in a reference subject or a reference population. Like the transcript level of said marker gene for the test sample, it therefore corresponds to the quantity of one or more transcripts of said marker gene or to a derived value which is representative of the quantity of transcripts of said marker gene, but in a reference sample of a reference subject or in a set of reference samples (for a population of reference subjects). The methods for determining the transcript level for the test sample and for prior determination of a reference level are equivalent or identical. Thus, a reference sample is of the same type as the test sample, namely a sample from the mouth or nose of a subject and, preferably, an oropharyngeal or nasopharyngeal sample, or saliva.

A reference subject may be a subject not infected with a replicating respiratory virus, i.e. notably a subject detected positive for the presence of a respiratory virus but for which the virus is not replicative, or a subject not infected with a respiratory virus (notably not positive in a test for detecting a respiratory virus); a subject not infected with a respiratory virus (in particular not positive to a test for detecting a respiratory virus); a healthy subject, i.e. with no infection whatsoever, as is notably the case with blood donors.

The reference sample (from a “reference” subject) for determining a reference level used as a threshold value for the transcript level of a marker gene is, advantageously, a sample from a subject (called “reference subject”) not infected with a replicating respiratory virus, i.e. notably a subject detected positive for the presence of a respiratory virus but for which the virus is not replicative, or a subject not infected with a respiratory virus (notably not positive in a test for detecting a respiratory virus), or a mixture of such samples.

The reference sample (from a “reference” subject) for determining the reference level used for calculating a ratio or derived value or score as previously described is, advantageously, a sample from a subject (called “reference subject”) not infected with a respiratory virus (in particular not positive to a test for detecting a respiratory virus), or a mixture of such samples.

It may also be verified that the reference subjects from which the reference samples come do not have neutralizing autoantibodies directed toward interferon α and/or do not have neutralizing autoantibodies directed toward interferon ω. Such verification may be carried out based on a blood sample, notably the serum of the subject concerned, using any known antibody detection technique, notably an ELISA technique. The reference sample may also be a sample of such a subject, treated ex vivo with an agent stimulating the immune system (such as LPS or lipopolysaccharide). The reference sample may also be a mixture of untreated sample(s) and sample(s) treated ex vivo with an immune system stimulating agent.

As regards the reference transcript level, when it is the transcript level of a reference population, it may be the mean or median of the different transcript levels measured for said population, i.e. the mean or median of the transcript levels measured in the reference samples.

The method according to the invention may also involve detecting the DNA or RNA of one or more respiratory viruses, and in particular SARS-COV-2 or one of its variants (Mommert, M. et al., 2020). Such detection techniques are known to a person skilled in the art. It is possible to use a non-quantitative, quantitative or semi-quantitative detection technique. In particular, an amplification, hybridization or sequencing technique adapted to the DNA or RNA of the desired virus may be used.

A subject of the invention is also a kit comprising means for determining the transcript level of one or more ISG marker genes as defined in the context of the invention. Such means make it possible to determine quantitatively the transcript level of said gene selected in the test sample. As explained previously, the transcript level determined may not be equal to the quantity of transcripts present in the test sample, but may be a derived value representing the amount of transcripts present in the test sample. Classically, the determination may indeed include a step of amplification and/or normalization and/or calculation of a ratio.

The term “kit” means a set of products and/or tools to be used together to obtain, in particular, the determination of the transcript level of one or more ISG marker genes as defined within the context of the invention. The products or tools required may or may not be grouped together in the same kit or device.

Thus, the kits according to the invention may comprise amplification and/or detection means for one or more ISG marker genes, such as those previously mentioned, in particular chosen from IFI27, IFI44L, IFIT1, RSAD2, ISG15 and SIGLEC1. The kits according to the invention may comprise amplification and/or detection means for each ISG marker gene selected, and in particular which are specific to said marker gene. Moreover, the kits according to the invention may also comprise at least one determination means, making it possible to determine the transcript level of a housekeeping gene. Thus, the kits according to the invention may comprise amplification and/or detection means for one or more housekeeping genes (preferably selected from the list consisting of: DECR1, HPRT1 and PPIB). Here again, the kits according to the invention may comprise amplification and/or detection means for each housekeeping gene selected, and in particular which are specific to said housekeeping gene.

The means for determining the transcript level comprise, notably, one or more amplification products or tools and/or one or more detection products or tools. In particular, for each marker gene or housekeeping gene whose transcript level is to be determined, the kit comprises one or more oligonucleotides for amplifying and/or detecting a transcript of said gene, notably an amplification primer or pair of primers and/or at least one probe for detecting a transcript of said gene.

The term “primer” or “amplification primer” means an oligonucleotide or nucleotide fragment which may consist of 5 to 100 nucleotides, preferably 15 to 30 nucleotides, and which has a specificity for hybridization with a target nucleotide sequence, under conditions determined for the initiation of an enzymatic polymerization, for example in an enzymatic amplification reaction of the target nucleotide sequence. In general, “primer pairs” are used, consisting of two primers. When it is desired to perform amplification of several different genes, several different primer pairs are preferably used, each preferentially having the ability to hybridize specifically with a different gene.

The term “probe” or “hybridization probe” means an oligonucleotide or nucleotide fragment typically consisting of 5 to 100 nucleotides, preferably 15 to 90 nucleotides and even more preferably 15 to 35 nucleotides, having a hybridization specificity under defined conditions to form a hybridization complex with a target nucleotide sequence. The probe also includes a reporter (such as a fluorophore, an enzyme or any other detection system), which will enable the target nucleotide sequence to be detected. In the present invention, the target nucleotide sequence may be a nucleotide sequence comprised in a messenger RNA (mRNA) or a nucleotide sequence comprised in a complementary DNA (cDNA) obtained by reverse transcription of said mRNA. When it is desired to target several different genes, several different probes are preferably used, each preferentially having the ability to hybridize specifically with a different gene.

Primer and probe sequences adapted to determine the transcript level of each gene IFI27, IFI44L, IFIT1, RSAD2, ISG15 and SIGLEC1 are notably described in M. Bergallo et al., 2020.

The term “hybridization” means the method whereby, under appropriate conditions, two oligonucleotides or nucleotide fragments, such as, for example, a hybridization probe and a target nucleotide fragment, having sufficiently complementary sequences, are capable of forming a double strand with stable and specific hydrogen bonds. A nucleotide fragment “capable of hybridizing” with a polynucleotide is a fragment which may hybridise with said polynucleotide under hybridization conditions, which may be determined in each case in a known manner. The hybridization conditions are determined by the stringency, i.e. the harshness, of the operating conditions. The higher the stringency, the more specific the hybridization. Stringency is defined notably as a function of the base composition of a probe/target duplex, and also by the degree of mismatch between two nucleic acids. Stringency may also be a function of reaction parameters, such as the concentration and type of ionic species present in the hybridization solution, the nature and concentration of denaturing agents and/or the hybridization temperature. The stringency of the conditions in which a hybridization reaction must be performed will depend mainly on the hybridization probes used. All these data are well known and the appropriate conditions may be determined by a person skilled in the art. In general, depending on the length of the hybridization probes used, the temperature for the hybridization reaction is between about 20 and 70° C., in particular between 35 and 65° C. in a saline solution at a concentration of about 0.5 to 1 M. A step is then performed to detect the hybridization reaction.

The term “enzymatic amplification reaction” refers to a method generating multiple copies of a target nucleotide fragment by the action of at least one enzyme. Such amplification reactions are well known to a person skilled in the art and mention may notably be made of the following techniques: PCR (Polymerase Chain Reaction), LCR (Ligase Chain Reaction), RCR (Repair Chain Reaction), 3SR (Self Sustained Sequence Replication) with patent application WO-A-90/06995, NASBA (Nucleic Acid Sequence-Based Amplification), TMA (Transcription Mediated Amplification) with U.S. Pat. No. 5,399,491, and LAMP (Loop mediated isothermal amplification) with U.S. Pat. No. 6,410,278. When the enzymatic amplification reaction is a PCR, it is more particularly referred to as RT-PCR (RT for “reverse transcription”), when the amplification step is preceded by a step of reverse-transcription of messenger RNA (mRNA) into complementary DNA (cDNA), and as qPCR or RT-qPCR when the PCR is quantitative.

The kits according to the invention may comprise means for detecting a respiratory virus, preferentially SARS-COV-2. In particular, such means correspond to a product or tool for detecting and/or amplifying the DNA or RNA of said virus.

The kits according to the invention may also include positive control means, notably a positive control sample, making it possible to qualify the quality of the RNA extraction, the quality of any amplification and/or hybridization method.

According to certain embodiments, the kits according to the invention, whatever their embodiment, are characterized in that they comprise a set of amplification and/or detection means which allow the detection and/or amplification of at most 100 genes, preferably at most 90, preferably at most 80, preferably at most 70, preferably at most 60, preferably at most 50, preferably at most 40, preferably at most 30, preferably at most 20, preferably at most 10 genes, notably at most 6, at most 5, at most 4, at most 3, or at most 2 genes, in total. The genes include virus genes, notably respiratory viruses, and also “biomarker” genes, i.e. an objectively measurable biological feature that represents an indicator of normal or pathological biological methods or of pharmacological response to a therapeutic intervention. Biomarker genes therefore include marker genes selected from the ISGs and housekeeping genes described in the context of the invention. The biomarker gene may, in particular, be detectable at mRNA level. Other examples of biomarkers that may also be present in a kit according to the invention include an endogenous biomarker or loci (such as a gene or an HERV/Human Endogenous RetroVirus) found in the chromosomal material of an individual, or an exogenous biomarker (such as a virus). According to particular embodiments, the kits according to the invention comprise, as gene amplification and/or detection means, exclusively biomarker gene amplification and/or detection means consisting exclusively of means for amplifying and/or detecting one or more ISG marker genes, means for amplifying and/or detecting one or more housekeeping genes and, optionally, means for amplifying and/or detecting one or more respiratory viruses.

Said products or means of determination, more precisely, said amplification products and/or detection products may be bound to the same solid support. The kit may therefore comprise a solid support comprising one or more oligonucleotides suitable for determining the transcript level of the or each IGS marker gene, or even one or more oligonucleotides suitable for determining the transcript level of the or each selected housekeeping gene, or even one or more oligonucleotides suitable for detecting a respiratory virus such as SARS-COV-2, as previously described. Such solid supports are well known to those skilled in the art, and notably described in applications WO 2008/140568 and WO 2017/093672 to which reference may be made for more details.

The kits are intended to be used in an automated manner with regard to comparison with a threshold value and/or reference value. In particular, the kits are intended to be used in an automated manner with regard to the comparison of the transcript level(s) of the selected ISG marker gene(s) and the threshold value(s) used, or where appropriate by using a reference level to calculate data (or a score) from the transcript level(s) of the selected ISG marker gene(s) and compare it with a threshold value. Furthermore, advantageously, they comprise, in addition to one or more threshold values, at least one reference level for said (or each) selected ISG marker gene, which is either stored on a computer-readable medium, or intended to be used in the form of a computer-executable code, notably configured to compare the transcript level of the ISG marker gene determined for the test sample, with said reference level.

A subject of the invention is also the use of a kit as described in the context of the invention to determine whether a subject is infected with a replicating respiratory virus.

In particular, the use will be directed toward determining the presence or absence in a subject of a replicating respiratory virus chosen from replicative seasonal coronaviruses, replicative SARS-COV-2 viruses (whatever the variant concerned), influenza viruses (influenza A, B and C), respiratory syncytial virus (RSV), rhinoviruses, metapneumoviruses, parainfluenza viruses and adenoviruses. The examples show, notably, that the kits according to the invention are particularly suitable for determining the presence or absence in a subject of a replicating respiratory virus chosen from replicative SARS-COV-2 viruses, whatever the variant concerned, and the influenza virus.

The specific or preferred embodiments described in relation to the methods according to the invention apply, of course, to the kits and uses which are the subject of the invention.

In the context of the invention, advantageously, the in vitro or ex vivo determination of the presence in a subject of an infection with a replicating respiratory virus is based solely on the determination, in a test sample from the mouth or nose of said subject, of the transcript level of one or more marker genes selected from among the interferon-stimulated genes, known as ISGs. According to a particular embodiment illustrated in the examples, only the levels of one or more marker genes selected from among the interferon-stimulated genes, known as ISGs, are determined and used as marker genes. In other words, the ISG genes are the only markers used to draw a conclusion about the presence or absence of infection with a replicating respiratory virus in the subject. It is, however, possible, although not preferred, to use, in addition to the marker gene(s) selected from the interferon-stimulated genes, known as ISGs, one or more other marker genes, for example, selected from the type I interferon genes. As examples of additional marker gene(s), mention may be made of the genes encoding the various proteins of type I, II and/or Ill interferons.

The invention also relates to methods for determining in vitro the presence in a subject of an infection with a replicating respiratory virus, as defined in the present description, which also comprise treatment of infection with a respiratory virus. In particular, treatment is initiated once it is concluded that said subject is infected with a replicating respiratory virus. Treatment may also be initiated once it is concluded that said subject is infected with a replicating respiratory virus and a respiratory virus present has been identified. In this case, treatment will be directed toward the respiratory virus identified. Even if there is no certainty that the respiratory virus whose presence has been identified and the respiratory virus present which is replicative are identical, the presumption is strong because the probability of the subject being infected by two different respiratory viruses is particularly reduced. According to another variant, treatment may be initiated as soon as the subject presents symptoms of a respiratory infection and it is concluded that said subject is infected with a replicating respiratory virus.

The treatment consists in administering a suitable antiviral drug. Antiviral treatments that may be mentioned include Iopinavir®, Ritonavir®, recombinant interferons, notably interferon beta, alpha and lambda. Numerous therapeutic treatments for COVID-19 are currently being tested (Canedo-Marroquín, G. et al., 2020).

The invention also relates to methods for determining in vitro the presence in a subject of an infection with a replicating respiratory virus, as defined in the present description, which also comprise the implementation of measures involving isolation, confinement and/or the obligation to wear a mask for said subject. Such measures may be put in place pending the outcome of the determination of infection with a replicating respiratory virus, and may be lifted if it is concluded that infection with a replicating respiratory virus is not present in said subject. It is also possible for such measures to be put in place if it is concluded that said subject is infected with a replicating respiratory virus.

The invention also relates to a method for determining, in vitro, the presence in a subject of an infection with a replicating respiratory virus, as defined in the present description, which comprises, in addition to the steps previously described, implementing, or not implementing, or stopping measures involving isolation, confinement and/or the obligation to wear a mask for said subject, depending on the conclusion reached by said method as to the presence or absence of an infection with a replicating respiratory virus in said subject.

According to particular embodiments, the methods according to the invention, whatever their embodiments, may also include a subject management step, depending on the result given by the method. A subject identified as having a respiratory infection with a replicating virus, and in particular with SARS-cov-2, may be subjected to a treatment protocol or to measures involving isolation, confinement and/or the obligation to wear a mask.

The present invention is illustrated in a non-limiting manner by the following examples.

### Example 1: Respiratory Replicating Virus SARS-COV-2

### Materials and Method

### Participants

Table 1 hereinbelow summarizes the demographics and key clinical characteristics of the study cohort: healthcare workers (HCWs) with mild symptoms of COVID-19 (mild COVID-19) and patients with critical COVID-19 admitted to an intensive care unit (ICU) (critical COVID-19). HCW with mild COVID-19 were not hospitalized and were free of pneumonia.

TABLE 1

| | |
|---|---|
| Benign | Critical |
| COVID-19 | COVID-19 |
| n = 44 | n = 19 |

Demographics

| Age, median [IQR] | | 38 | [28.8-48] | 59 | [54-68.5] |
|---|---|---|---|---|---|
| Gender | Male, n (%) | 5 | (11) | 14 | (74) |
| | Female, n (%) | 39 | (89) | 5 | (26) |

| | | | | |
|---|---|---|---|---|
| BMI, median [IQR] | 23.9 | [22.4-26.3] | 30.4 | [25.4-35.6] |

Clinical characteristics

| Time from onset of symptoms to ICU | N/A | 8 | [6-12] |
|---|---|---|---|
| admission, Day, median [IQR] | | | |
| Length of stay in ICU, Day, median | N/A | 11 | (4-69) |
| (min-max) | | | |

| | | | |
|---|---|---|---|
| Mortality, n (%) | N/A | 6 | (32%) |

Abbreviations: BMI, body mass index; IQR, interquartile range; N/A, not applicable

More information regarding the cohort is described below.

Participants with Benign COVID-19

A prospective longitudinal cohort study was performed at the University Hospital of Lyon, France (Hospices Civils de Lyon, HCL), including HCW with symptoms suggestive of SARS-COV-2 infection (at least one of the following symptoms: fever, respiratory symptoms, headache, anosmia, ageusia-Trouillet-Assant, S. et al. 2020a). The diagnosis of COVID-19 was confirmed in all patients by qRT-PCR (Cobas® SARS-COV-2 test, Roche Diagnostics, Basel, Switzerland). The nasopharyngeal (NP) swab used was either in Copan universal transport medium (UTM-RT®) or in a tube of Cobas® PCR medium. Clinical and microbiological data were collected for all HCW included in the study. Patients with a positive RT-PCR result at inclusion (V1) returned weekly (V2, V3 and V4) for blood and nasopharyngeal sampling until RT-PCR was negative. Written informed consent was obtained from all participants; ethics committee approval was obtained from the National Review Board for Biomedical Research in April 2020 (Comité de Protection des Personnes Sud Méditerranée I, Marseilles, France; ID RCB 2020-A00932-37), and the clinical study was registered on ClinicalTrials.gov (NCT04341142).

Patients with Critical COVID-19

The NP samples used to perform the SARS-COV-2 RT-PCR detection test during hospitalization were collected and used for the various additional experiments. During the ICU stay, blood collected in EDTA tubes was used to test for anti-IFN antibodies. All critically ill patients admitted to the ICU were included in the MIR-COVID study. This study was registered with the French National Data Protection Agency under number 20-097 and was approved by a biomedical research ethics committee (Comité de Protection des Personnes HCL) under number No. 20-41. In accordance with the General Data Protection Regulation (Regulation (EU) 2016/679 and Directive 95/46/EC) and the French Data Protection Act (Law No. 78-17 of 6 Jan. 1978 and Decree No. 2019-536 of 29 May 2019), the informed consent of each patient or their next of kin has been obtained.

### FilmArray® for the Determination of ISG Transcripts

The FilmArray® prototype (in the form of a pouch) which was used makes it possible to determine the transcript levels of four ISGs (interferon alpha-inducible protein 27 (IFI27), interferon-induced protein 44 like (IFI44L), interferon-induced protein with tetratricopeptide repeats (IFIT1), and radical S-adenosyl methionine domain-containing protein 2 (RSAD2)) and three housekeeping genes (hypoxanthine phosphoribosyl transferase 1 (HPRT1), peptidylpropyl isomerase B (PPIB), 2,4-dienoyl-CoA reductase 1 (DECR1)), for signal normalization. One hundred microlitres of NP sample or PAXgene™ blood were tested with the IFN prototype (Tawfik, D. M. et al., 2020) in accordance with the manufacturer's instructions. Briefly, the pouches were hydrated with the hydration solution provided with the kit. NP or PAXgene™ blood samples were mixed with 800 μl of the sample buffer provided with the kit and directly injected into the pouch and run on FilmArray® 2.0 and FilmArray® Torch instruments (BioFire Diagnostics, LLC., Salt Lake City, UT). Results were delivered in less than an hour. Using a research version of the instrument, real-time quantification cycle (Cq) values and post-amplification melting curves were measured for each assay. Normalized transcript values for each assay were then calculated using the housekeeping genes. An ISG score in the case of NP samples was calculated using the same method as applied to PAXgene samples, as previously described (Pescarmona, R. et al., 2019).

### Protein Measurement of IFN-α and IFN-2

Serum IFN-α2 concentrations (fg/ml) from plasma were determined with an ultrasensitive assay (SIMOA®) using a commercially available kit for IFN-α2 quantification (Quanterix™, Lexington, MA). The assay was based on a three-step protocol using an HD-1 analyser (Quanterix) and required a processing time of 3 hours from sampling to results. Interferon lambda was determined with an assay using a commercially available kit for the quantification of IFN-2 (mesoscale discovery).

### Measuring Viral Load

SARS-COV-2 load was determined from an NP sample using the SARS-COV-2 R-gene® kit (bioMérieux, Lyon, France). Briefly, nucleic acid extraction was performed from 0.2 ml of an NP sample on NUCLISENS® easyMAG® and amplification using a Bio-Rad CFX96 thermal cycler. Viral load was quantified using four in-house developed quantification controls (QS) targeting the SARS-CoV-2 N gene: QS1 to QS4 at 2.5×10⁶, 2.5×10⁵, 2.5×10⁴, 2.5×10³ copies/ml of SARS-COV-2 control DNA, respectively. These QSs were monitored and quantified using a Nanodrop spectrophotometer (ThermoFisher) and Applied Biosystems QuantStudio 3D digital PCR. In parallel, NP samples were tested with the CELL Control R-GENE® kit (amplification of the domestic HPRT1 gene) which contains 2 quantification controls QS1 and QS2, respectively at 10⁴ copies/μl (50 000 cells/PCR i.e. 1.25×10⁶ cells/ml under the conditions of the examples) and 10³ copies/μl (5000 cells/PCR i.e. 1.25×10⁵ cells/ml under the conditions of the examples) of control DNA, to normalize the viral load according to the quality of the sampling (Log 10 [(SARS-COV-2 copy number per ml/number of cells per ml)×10⁶ cells per ml]).

### Viral Culture

Viral culture was performed in accordance with WHO draft biohazard prevention guidelines [WHO/WPE/GIH/2020.3] using NP samples in UTM-RT®. RT-PCR positive NPs were inoculated onto confluent Vero cells (ATCC CCL-81@) with Eagle's minimum essential medium (EMEM) supplemented with 2% penicillin-streptomycin, 1% L-glutamine, and 2% inactivated fetal calf serum. The plates were incubated at 33° C. with 5% CO₂ for 96 hours. Cytopathic effects (CPE) were monitored daily; samples were harvested when positive, while samples negative at 96 hours were subcultured onto new plates. Culture supernatants were sampled 2 hours after inoculation, after 96 hours, and again after 96 hours in subculture. RNA from supernatants was extracted by an MGISP-960 automated workstation using the MGI Easy Magnetic Bead Viral DNA/RNA Extraction Kit (MGI Tech©, Marupe, Latvia), and detection of SARS-CoV-2 was performed using the TaqPath™ COVID-19 CE-IVD RT-PCR Kit on a QuantStudio™ 5 system (Applied Biosystems, Thermo Fisher Scientific, Waltham, USA).

### Serological Test

The presence of anti-SARS-COV-2 antibodies was assessed using the Wantai Ab assay, which detects total antibodies directed toward the receptor-binding domain (RBD) of protein S.

The presence of anti-IFN-α antibodies was investigated using a commercially available kit (Thermo-Fisher) (David, G. et al., 2021). The ability of patient serum to neutralise IFN-α was then assessed as previously described (Bastard, P. et al., 2021).

### Statistical Analysis

Non-parametric Wilcoxon-Mann-Whitney tests and Spearman correlation were performed for all comparisons, unless otherwise stated. To calculate the viral load threshold that best divided patients into two ISG groups, the minimum p-value of the non-parametric significance test (Wilcoxon rank sum) was used, applied iteratively to the ISG score, separated on normalized nasal viral load thresholds ranging from minimum (0.60) to maximum (9.00) detected values with a step size of 0.01. All statistical analyses were performed using R (R Foundation, https://www.r-project.org/foundation/version 3.6.1) and GraphPad Prism 8.3.0 software. A p-value <0.05 was considered statistically significant. Correlations were only performed on samples where double detection had been possible.

### Results

### Coordinated Regulation of Nasal Passage and Blood ISGs in Response to IFN-I During Infection

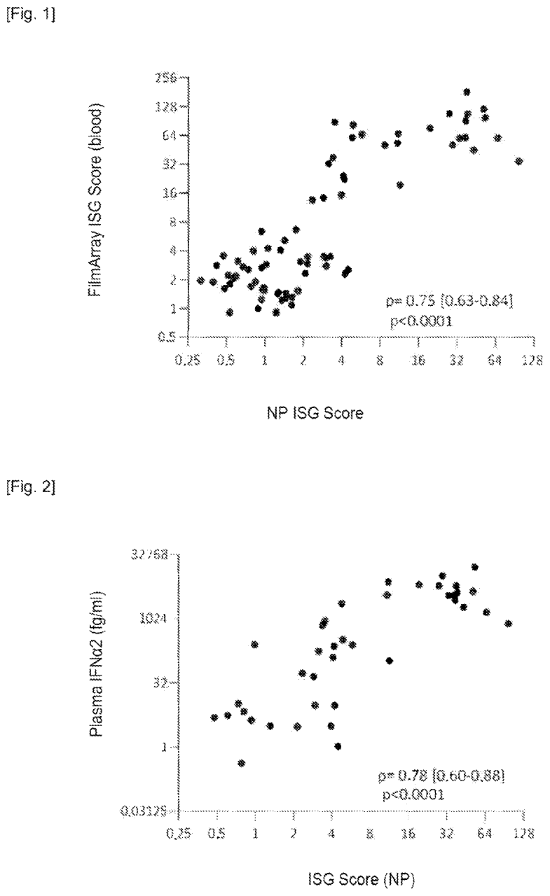

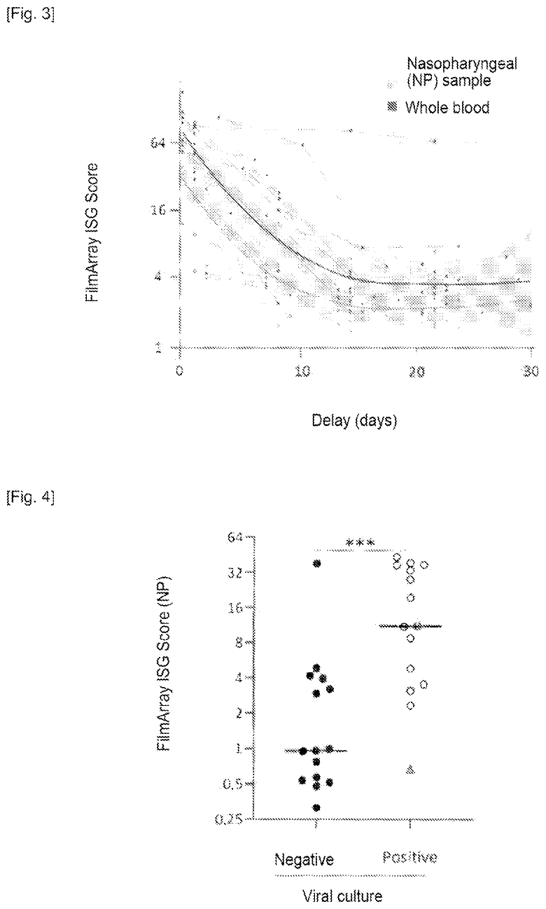

The regulation and role of type I and III interferon signalling was studied in the nasal mucosa in response to SARS-COV-2 infection. To this end, nasopharyngeal samples used for RT-qPCR detection of SARS-COV-2 were used. However, as the quantity of cellular material and transcripts present for this type of sample was very limited, FilmArray® technology was used. This PCR technique, already described in the literature (Mommert, M. et al., 2020), enables rapid and sensitive measurement of the transcript levels of various ISGs and was adapted to nasopharyngeal samples. The advantage of this technique is that it is semi-automated and also designed for clinical routine, giving a score derived from the quantification of four ISGs regulated via IFN-stimulated gene factor 3 (ISGF3)-dependent ISRE elements, namely IFI27, IFI44L, RSAD2 and IFIT1 (Hernandez, N. et al., 2018). This score was analyzed during diagnosis in 23 HCW infected with SARS-COV-2, and compared to the same score obtained from blood samples and serum IFN-α2 level from the same patients. A strong correlation was observed between scores obtained from nasal-pharyngeal (NP) and blood samples and between scores obtained from nasal-pharyngeal samples and serum levels of IFN-α2 (Spearman ρ>0.75 for both comparisons, FIGS. 1 and 2). Serum levels of IFN-α1, on the other hand, did not correlate as well with scores obtained from nasal-pharyngeal and blood samples (results not shown). These results highlight the temporal association between antiviral responses based on ISG transcripts, at nasal and systemic levels, and suggest that IFN-α2 stimulates the generation of ISG transcripts measured in both cases. The evolution over time of the score in blood (called the blood score) and the score in nasal-pharyngeal samples (called the nasal score or NP score) was also studied. As shown in FIG. 3, the two parameters followed very similar kinetics, peaking at the earliest time points after the onset of symptoms and decreasing rapidly thereafter, then becoming virtually negative 14 days after the onset of symptoms. This correlation confirms the co-ordination of antiviral defences by IFN-I/III at nasal and systemic levels and shows that the nasal type I/III ISG score, measured using FilmArray® directly from NP samples, is an indicator that tracks the blood type I/III ISG score in patients.

The Score of the Transcript Levels of Different ISGs in Nasopharyngeal Samples Correlates with the Nasal Load of SARS-COV-2 and the Replication Capacity of the Virus.

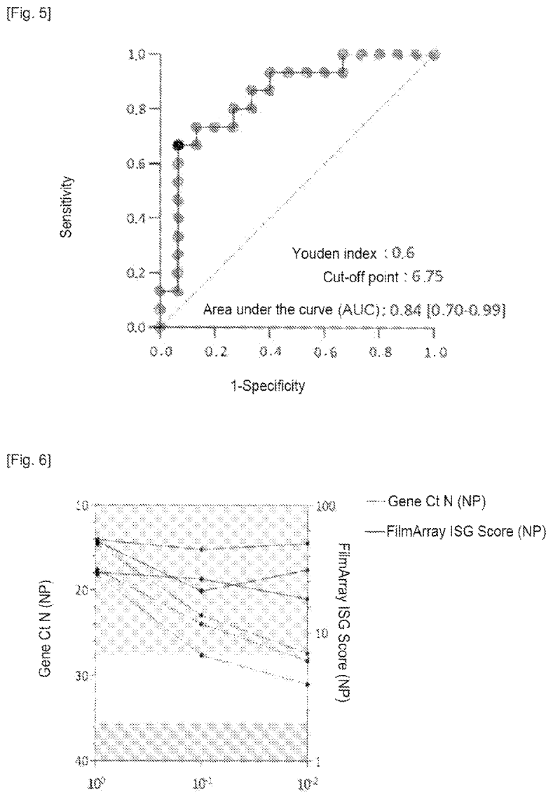

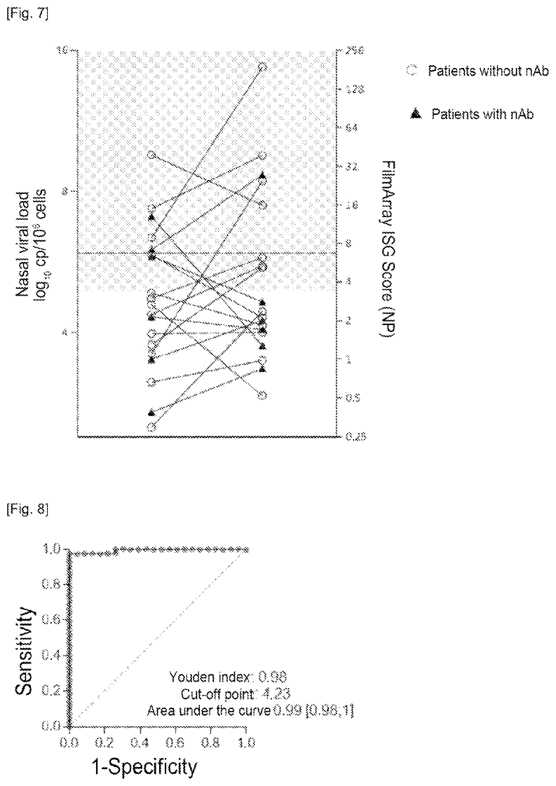

FIG. 7 shows that there is a significant correlation between the score associated with the transcript levels of the different type I/III ISGs (called the ISG score) obtained for the nasopharyngeal samples and the viral load. Directly from the swabs corresponding to the nasopharyngeal samples, cultures on Vero cells were performed, as previously described (Bal, A. et al., 2021), in order to assess the putative association between the ISG score in the nasopharyngeal samples and the replication capacity of the virus isolated from the swab. It was found that cultures producing a cytopathic effect on Vero cells (known as positive cultures) were predominantly associated with high ISG scores, while cultures not producing a cytopathic effect on Vero cells (known as negative cultures) were almost always associated with low ISG scores (FIG. 4). The value of using the ISG score as a marker indicative of infection with a replicating SARS-COV-2 virus was confirmed, using an ROC curve (FIG. 5). The area under the ROC curve [95% confidence interval] was 0.84 [0.7 to 0.99], showing a good performance of the ISG score in predicting viral replicative capacity with a separation score of 6.75. Finally, to further evaluate the ISG score as a marker of replicating virus infection, comparative measurements of the ISG score and viral RNA amounts under limited material conditions, mimicked by 1:10 serial dilutions of positive swabs, were performed. The results presented in FIG. 6 show that viral PCR Ct values increased rapidly following swab dilutions, while ISG scores remained relatively stable and consistently greater than the 6.75 value previously shown to distinguish nasopharyngeal samples containing or not containing the virus in its replicative state.

The type I/III ISG nasal score is lower in patients with neutralizing type I anti-IFN autoantibodies.

A fraction of severely affected Covid-19 patients have shown low or undetectable blood levels of type I IFN (Hadjadj, J. et al., 2020 and Trouillet-Assant, S. et al., 2020), which may be due either to congenital defects in type I IFN immunity (Zhang, Q. et al., 2020) or to the presence in the blood of autoantibodies which neutralise IFN-α or IFN-ω or both (Bastard, P. et al., 2020). A study was therefore conducted into the relationships between disease severity, the presence of autoantibodies directed toward a type I interferon, the viral load in nasopharyngeal samples (known as the nasal viral load), and the ISG score in nasopharyngeal samples (known as the nasal ISG score or the NP ISG score). For this purpose, a cohort of severely ill patients was assembled and blood samples and nasopharyngeal samples from nose swabs were obtained on ICU admission (n=19). Whereas 90% of patients with COVID-19 who were mildly symptomatic with a nasal ISG score greater than 6.75 (in accordance with the cut-off value defined by the Younden index in FIG. 5) had a normalized nasal viral load >4.90 Log₁₀ cp/10⁶ cells, only 57% of patients with critical COVID-19 (4 out of 7) with a similarly high nasal viral load had a nasal ISG score greater than 6.75. To explain this discrepancy between nasal viral load and nasal ISG score, the presence of neutralizing autoantibodies directed toward IFN-α and/or IFN-ω was determined in the serum of patients in this cohort using a new, highly sensitive Elisa assay (David, G. et al., 2021). Anti-IFN-α and/or anti-IFN-ω autoantibodies were detected in the serum of 6 out of 19 severely affected patients and in none of the mildly symptomatic patients (FIG. 7). Furthermore, patients with anti-IFN-I autoantibodies had very low nasal ISG scores, irrespective of viral load, except for one patient who had autoantibodies directed toward IFN-α2 only, and for whom it was assumed that IFN-ω compensated for IFN-α2 blockade. The remaining 5 patients had autoantibodies directed toward both a and w IFNs. These data suggest that neutralizing anti-IFN-I autoantibodies compromise early antiviral defences in the nasal mucosa of severely affected patients, particularly when autoantibodies directed toward both IFN-α2 and IFN-ω are present. These data also suggest that IFN-ω is involved in type I IFN-mediated immunity in the nasal passages.

### Example 2: Respiratory Replicating Influenza Virus

### Materials and Method

### Participants

Retrospective nasopharyngeal (NP) samples (2017-2018) from the Hospices Civils de Lyon (HCL) were selected based on the presence or absence of symptoms in the subjects, on confirmation of respiratory viral infection by influenza virus (Influenza B) RT-qPCR, and also on knowledge of the viral culture status. The clinical data of the patients from whom the samples were taken is shown in Table 2 hereinbelow.

TABLE 2

| | |
|---|---|
| Influenza virus | None (NEG) |
| n = 42 | n = 23 |

Age

| | | |
|---|---|---|
| Children <18 years, n (%) | 2 (4.8) | 2 (8.7) |
| Adults ≥18 years, n (%) | 40 (95.2) | 21 (91.3) |

Gender

| | | |
|---|---|---|
| Women, n (%) | 20 (47.6) | 7 (30.4) |
| Men, n (%) | 22 (52.4) | 16 (69.6) |

Post-symptom delay (PSD)

| | | |
|---|---|---|
| ≤10 days, n (%) | 39 (92.8) | 0 (0) |
| >10 days, n (%) | 1 (2.4) | 0 (0) |
| Unknown, n (%) | 2 (4.8) | 0 (0) |
| No symptoms, n (%) | 0 (0) | 23 (100) |

Viral culture

| | | |
|---|---|---|
| Negative, n (%) | 11 (26.2) | — |
| Positive, n (%) | 31 (73.8) | — |

Co-pathogen detection

| | | |
|---|---|---|
| Viruses, n (%) | 0 (0) | 0 (0) |

NP swabs from healthy volunteers (HV) were recruited using the same protocol as for example 1. The RESPIFERON study was approved by the Ethics Committee for Biomedical Research and by the CPP Committee for the Protection of Individuals. Authorization to use the samples was granted on the basis of the non-objection of the subjects directly concerned or their legal guardians, where applicable, after informing them in writing. Anonymization of the subjects' personal data was performed prior to their transfer to the laboratory.

### FilmArray® for the Determination of ISG Transcripts

The materials and methods used in this section are identical to those used in Example 1.

### Measuring Viral Load

Viral load was quantified for each NP sample. Nucleic acids were extracted using the NucliSens® easyMAG™ automated extractor (bioMérieux, Marcy l′Etoile). The extraction was performed using 200 μL of NP samples which were deposited in lysis buffer. After a 10 min incubation at room temperature, 50 μL of magnetic silica was added before starting the extraction.

On conclusion of the extraction, 50 μL of eluate containing the extracted RNA were recovered. The extracted RNA was reverse transcribed and amplified using the ready-to-use Influenza A/B R-gene® real-time RT-PCR kit. The reaction required 10 μL of eluate, 15 μL of amplification premix containing the reagents required for PCR and 0.15 μL of reverse transcriptase (diluted 10-fold).

Amplification was run on the Bio-Rad CFX96 following a specific protocol, namely 5 min at 50° C. for reverse transcription followed by 15 min at 95° C. to activate the Taq polymerase, and finally 45 alternating cycles: 10 sec at 95° C. for denaturation, 40 sec at 60° C. for hybridization, and 25 sec at 72° C. for elongation.

Viral load was quantified using a range of r-gene standards (QS1 to QS4 containing 10⁵, 10⁴, 10³ and 10² copies/μL of plasmid respectively). In parallel, the CELL Control R-gene® kit was used to check for the presence of cells in the sample and to quantify them by detecting the HPRT1 housekeeping gene, and also to normalize the viral load. This kit contains two quantification standards, QS1 and QS2 at 10⁴ and 10³ copies/μL DNA respectively. Data were recovered, processed and analyzed using CFX Maestro software. Viral load was normalized by dividing the viral RNA copy number/PCR by the cell number/PCR and is expressed as log 10 of the copy number per 10⁶ cells.

### Viral Culture

Virus culture, sample collection, sampling of culture supernatants and extraction of RNA from said supernatants are as described in example 1. Detection of the influenza virus was performed using the Influenza A/B R-gène® kit (bioMérieux, Marcy l′Etoile).

### Statistical Analysis

The materials and methods used in this section are identical to those used in Example 1.

### Results

Influenza Virus Infection is Associated with an Increased Nasal IFN Response

To assess the relationship between nasal IFN I/III response and influenza virus infection, for each NP sample included, an IFN I/III score based on the expression of four ISGs in the nasal mucosa measured by RT-qPCR using the BioFire® IFN FilmArray® pouch prototype was calculated.

This nasal IFN I/III score, which is informative as regards the local IFN response, was found to be significantly higher (p<0.0001) for PCR-positive samples, i.e. from individuals infected with the influenza virus, compared with PCR-negative samples corresponding to uninfected individuals.

In addition, a Receiver Operating Characteristic (ROC) curve was constructed to determine the ability of the nasal IFN I/III score to discriminate between viral negative PCR and viral positive PCR samples. FIG. 8 shows that the IFN I/III score performed very well in predicting the presence of viral infection, with an area under the curve of 0.98 and a cut-off point of 3.29 (characterized by 93% sensitivity and 100% specificity).

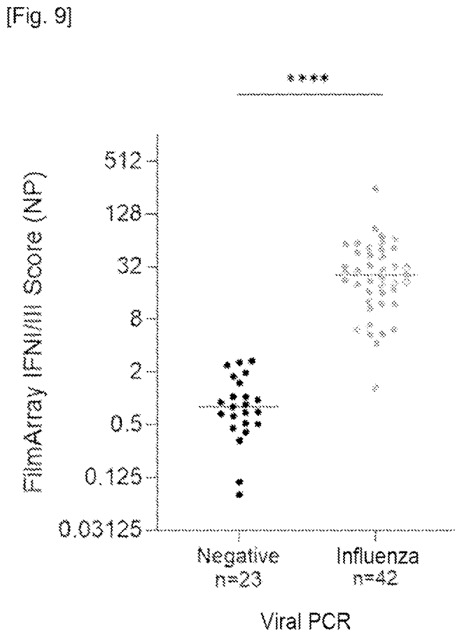

As shown in FIG. 9, the same observation could be made by analysing the IFN I/III score for influenza virus infection. Taken together, the results suggest that the nasal IFN I/III response discriminates between infected and uninfected subjects, thus potentially reflecting the presence of any respiratory viral infection.

### ISG Transcriptome Signature in Infection

The level of ISG mRNA expression, expressed as log 2 RQ ratios, was estimated for each NP sample from the results obtained with the IFN FilmArray® BioFire® pouch prototype as previously explained (see materials and methods).

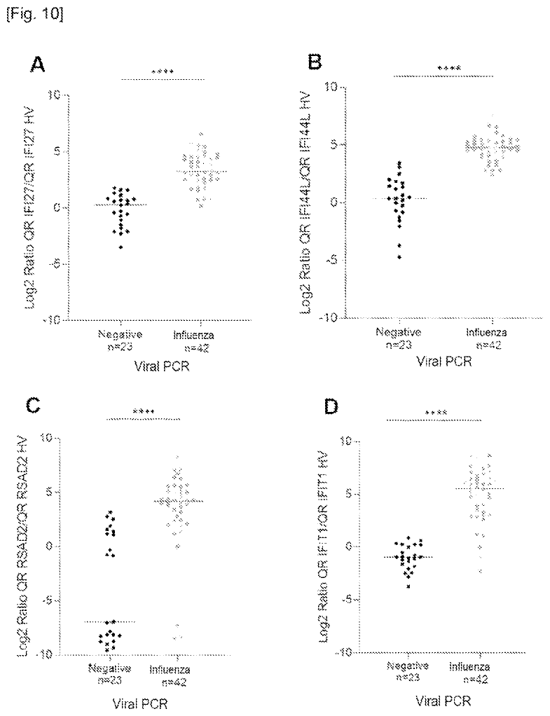

The results presented in FIG. 10 clearly show that the expression levels of the IFI27, IFI44L, RSAD2 and IFIT1 transcripts were significantly higher in samples from subjects infected with the influenza virus than in samples from uninfected subjects, thus confirming the involvement of interferon-stimulated genes in the host response to infection by the virus.

### Nasal INF Response Reflects Active Influenza Virus Infection

The nasal IFN response of NP samples from subjects infected with the influenza virus whose viral culture status (positive or negative) was known, was analyzed.

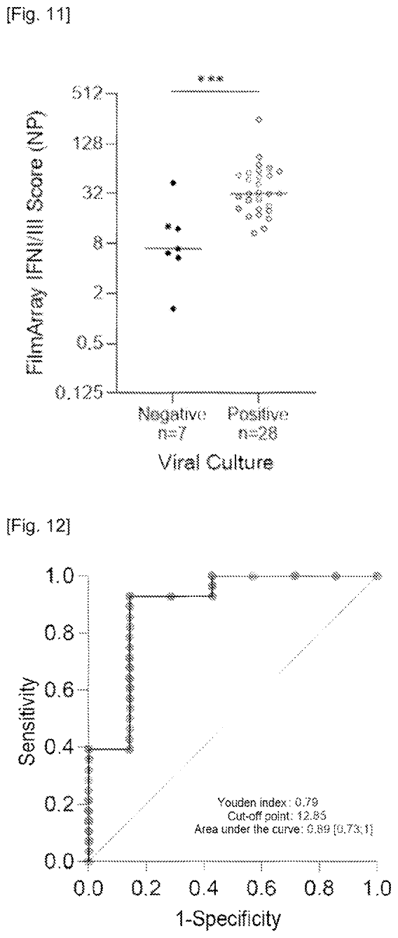

As shown in FIG. 11, when comparing the nasal IFN I/III score calculated using FilmArray® technology according to viral culture status, it was found that the score was significantly higher for the positive viral culture samples than for the negative viral culture samples in the case of influenza virus infection.

To confirm the relevance of the IFN I/III score for use as a biomarker of the infectious nature of the influenza virus, an ROC curve was established. FIG. 12 thus highlights the good performance of the IFN I/III score in predicting the replicative capacity of the GRB virus, with an area under the curve of 0.89 and a cut-off point of 16.08, for a sensitivity of 0.93 and a specificity of 0.86.

Together with the preceding example, this example confirms that measuring the transcript level of interferon-stimulated genes, known as ISGs, effectively and reliably detects the presence of infection with a replicating respiratory virus, for instance SARS-COV-2 or the influenza virus, thus providing a new tool for clinicians to rapidly identify patients presenting a risk of viral transmission, and alternatively to avoid quarantine measures for patients who are not, or are no longer, a possible source of contamination.

### REFERENCES

Trouillet-Assant, S., et al., Assessment of serological techniques for screening patients for COVID-19 (COVID-SER): a prospective, multicentric study. BMJ Open 10, e041268 (2020a).
Tawfik, D. M., et al., Immune Profiling Panel: A Proof-of-Concept Study of a New Multiplex Molecular Tool to Assess the Immune Status of Critically Ill Patients. J. Infect. Dis. 222, S84-S95 (2020).
Pescarmona, R., et al., Comparison of RT-qPCR and Nanostring in the measurement of blood interferon response for the diagnosis of type I interferonopathies. Cytokine 113, 446-452 (2019).
David, G., et al., Antibodies against type-I Interferon: detection and association with severe clinical outcome in COVID-19 patients. medRxiv 2021.04.02.21253262 (2021) doi:10.1101/2021.04.02.21253262.
Bastard, P., et al., Auto-antibodies to type I IFNs can underlie adverse reactions to yellow fever live attenuated vaccine. J. Exp. Med. 218 (2021).
Mommert, M., et al., Type-I Interferon assessment in 45 minutes using the FilmArray® PCR platform in SARS-COV-2 and other viral infections. Eur. J. Immunol. (2020) doi:10.1002/eji.202048978.
Hernandez, N., et al., Life-threatening influenza pneumonitis in a child with inherited IRF9 deficiency. J. Exp. Med. 215, 2567-2585 (2018).
Bal, A., et al., Six-month antibody response to SARS-COV-2 in healthcare workers assessed by virus neutralization and commercial assays. Clin. Microbiol. Infect. Off. Publ. Eur. Soc. Clin. Microbiol. Infect. Dis. (2021) doi:10.1016/j.cmi.2021.01.003.
Hadjadj, J., et al., Impaired type I interferon activity and inflammatory responses in severe COVID-19 patients. Science 369, 718-724 (2020).
Trouillet-Assant, S., et al. Type I IFN immunoprofiling in COVID-19 patients. J. Allergy Clin. Immunol. (2020) doi:10.1016/j.jaci.2020.04.029.
Zhang, Q., et al., Inborn errors of type I IFN immunity in patients with life-threatening COVID-19. Science 370 (2020).
Bastard, P., et al., Autoantibodies against type I IFNs in patients with life-threatening COVID-19. Science 370 (2020).
Kricka, et al., Clinical Chemistry, n^{o} 45(4), p. 453-458 (1999).
Relier G. H., et al., DNA Probes, 2nd Ed, Stockton Press, sections 5 and 6, p. 173-249 (1993).
Mick, E., et al., Upper airway gene expression reveals suppressed immune responses to SARS-COV-2 compared with other respiratory viruses. Nat. Commun. 11 (2020).
Ng, D. L., et al., A diagnostic host response biosignature for COVID-19 from RNA profiling of nasal swabs and blood. Sci. Adv. 7 (2021).
Gupta, R. K., et al., Blood transcriptional biomarkers of acute viral infection for detection of pre-symptomatic SARS-COV-2 infection: a nested, case-control diagnostic accuracy study. Lancet Microbe 2021; 2:e508-17.
Han, M. S., et al., RT-PCR for SARS-COV-2: quantitative versus qualitative. Lancet Infect. Dis. 21, 165 (2021).
Leland D. S, et al., Role of Cell Culture for Virus Detection in the Age of Technology, Clinical Biology Reviews, vol. 20, N^{o} 1, p. 49-78 (January 2007).
Stelzer-Braid S., et al., Virus isolation of severe acute respiratory syndrome coronavirus 2 (SARS-COV-2) for diagnostic and research purposes, Pathology, 52(7), p. 760-763 (December 2020).
Monel B., et al., Release of infectious virus and cytokines in nasopharyngeal swabs from individuals infected with non-B.1.1.7 or B.1.1.7 SARS-COV-2 variants, (2021) https://doi.org/10.1101/2021.05.20.21257393.
Manry J., et al., Génétique des populations et immunité chez l'homme, Le cas des interférons. Med. Sci. (Paris) 28: p. 1095-1101, 2012.
Hertzorg P. J., et al., “New Interferons”, Encyclopedia of Immunobiology, Volume 2, p. 501-508, 2016.
Lazear, H. M., et al. Shared and Distinct Functions of Type I and Type III Interferons. Immunity 50, 907-923 (2019).
Hellemans, et al., Genome biology 8(2):R19 (2007).
Picard C, et al., Type I interferonopathies, Literature review. Rev. Med. Interne 39:271-278. (2018).
Bergallo M., et al., Interferon signature in immunosuppressed patients with lower respiratory tract infections: dosage on bronchoalveolar lavage, Minerva Medica; 111(3):245-53, (2020 June).
Canedo-Marroquín, G., et al., SARS-COV-2: Immune Response Elicited by Infection and Development of Vaccines and Treatments. Front. Immunol. 11, (2020).
Poritz M., et al., FilmArray, an automated nested multiplex PCR system for multi-pathogen detection: development and application to Respiratory tract infection, 2011, PLOS One 6(10): e26047. Doi: 10.1371/journal.pone.0026047

1. A method for determining in vitro or ex vivo the presence in a subject of an infection with a replicating respiratory virus, comprising a step i) of determining, in a test sample from the mouth or nose of said subject, the transcript level of at least one marker gene selected from interferon-stimulated genes, known as ISGs.

2. The method as claimed in claim 1, characterized in that in step i), the transcript level of one or more marker genes chosen from: IFI44L, IFIT1, IFI27, RSAD2, ISG15, and SIGLEC1, is determined.

3. The method as claimed in claim 1, characterized in that in step i), the transcript level of one or more marker genes chosen from: IFI44L, IFIT1, IFI27 and RSAD2, is determined.

4. The method as claimed in claim 1, characterized in that the test sample is an oropharyngeal or nasopharyngeal sample, or a saliva sample.

5. The method as claimed in claim 1, characterized in that step i) consists in determining the mRNA level of said marker gene(s).

6. The method as claimed in claim 1, characterized in that the test sample comes from a subject showing symptoms of an infection.

7. The method as claimed in claim 1, characterized in that the test sample comes from a subject who does not have neutralizing autoantibodies directed toward interferon α and/or neutralizing autoantibodies directed toward interferon ω.

8. The method as claimed in claim 1, characterized in that it comprises the following steps:
i) determining the transcript level of at least one marker gene chosen from the ISGs, for said test sample of the subject,
ii) comparing the transcript level of said at least one marker gene obtained, with a reference level.

9. The method as claimed in claim 1, characterized in that it also comprises a step iii) which is to conclude whether or not said subject is infected with a replicating respiratory virus, using at least one threshold value.

10. The method as claimed in claim 1, characterized in that it comprises the following steps:
i) determining the transcript level of a single marker gene selected from the ISGs, for the test sample,
ii) comparing the transcript level of said marker gene obtained, with a reference level, used as a threshold value, said reference level being the transcript level of said marker gene, in a subject not infected with a replicating respiratory virus, who preferably does not have neutralizing autoantibodies directed toward interferon α and/or neutralizing autoantibodies directed toward interferon ω, or in a population of such subjects and
iii) concluding that said subject is infected with a replicating respiratory virus if there is a difference with said reference level of said marker gene.

11. The method as claimed in claim 10, characterized in that said marker gene is IFIT1 or IFI44L.

12. The method as claimed in claim 1, characterized in that it comprises the following steps:
i) determining the transcript levels of several marker genes selected from the ISGs for the test sample, and
ii) comparing the transcript level of each marker gene determined in the test sample with a reference level corresponding to said marker gene, used as a threshold value, said reference level being the transcript level of said marker gene, in a subject not infected with a replicating respiratory virus, which preferably does not have neutralizing autoantibodies directed toward interferon α and/or neutralizing autoantibodies directed toward interferon ω, or in a population of such subjects, and
iii) concluding that said subject is infected with a replicating respiratory virus if there is a difference with the corresponding reference level for at least one of the marker genes for which the transcript level has been determined in the test sample.

13. The method as claimed in claim 1, characterized in that said difference corresponds to a transcript level of said marker gene determined for the test sample greater than that of said reference level.

14. The method as claimed in claim 1, characterized in that it comprises the following steps:
i) determining the transcript levels of several marker genes selected from the ISGs, for the test sample,
ii) comparing the transcript level of each marker gene determined in the test sample with a reference level corresponding to said marker gene, calculating the ratio between said transcript level and said reference level of said marker gene, said reference level preferably being the transcript level of said marker gene, in a subject not infected with a respiratory virus, or in a population of such subjects,
iibis) calculating the median of all the ratios obtained in step ii), and
iii) concluding that an infection with a replicating respiratory virus is present in said subject, when said median calculated in step iibis) is greater than a threshold value, which is preferably equal to 1.5.

15. The method as claimed in claim 1, characterized in that the determination of the transcript level of the marker gene(s) is performed by hybridization, amplification or sequencing, and, in particular, by RT-qPCR.

16. The method as claimed in claim 1, characterized in that the transcript level of the marker gene(s) obtained is a value normalized relative to the transcript level of one or more housekeeping genes, notably chosen from DECR1, HPRT1 and PPIB.

17. The method as claimed in claim 1, characterized in that, prior to or concomitantly with step i), the test sample is subjected to a diagnostic test, by detection of DNA or RNA, notably by PCR or RT-PCR, of a respiratory virus, notably chosen from the SARS-COV-2 viruses and the influenza virus.

18. The method as claimed in claim 17, characterized in that the test sample is subjected to a diagnostic test, by detection of DNA or RNA, notably by PCR or RT-PCR, of a respiratory virus chosen from the SARS-COV-2 viruses and the influenza virus, said diagnostic test preferably being performed prior to step i) and having given a positive result.

19. A kit for determining in vitro or ex vivo the presence in a subject of an infection with a replicating respiratory virus, comprising:
at least one means for determining the transcript level of a marker gene selected from ISGs, chosen from oligonucleotides,
at least one threshold value stored on a computer-readable medium, said threshold value corresponding to the transcript level of said marker gene in a subject who has no infection with a replicating respiratory virus and who, preferably, does not have neutralizing autoantibodies directed toward interferon α and/or does not have neutralizing autoantibodies directed toward interferon ω, or in a population of subjects who do not have any infection with a replicating respiratory virus and who preferably do not have neutralizing autoantibodies directed toward interferon α and/or do not have neutralizing autoantibodies directed toward interferon ω.

20. The kit as claimed in claim 19, characterized in that it also comprises at least one reference level for said marker gene, stored on a computer-readable medium, said reference level preferably being the transcript level of said marker gene in a subject not infected with a respiratory virus, or in a population of such subjects.

21. The detection kit as claimed in claim 19, characterized in that it also comprises at least one means for detecting a respiratory virus.

22. A kit for determining in vitro or ex vivo the presence in a subject of an infection with a replicating respiratory virus, comprising:
at least one means for determining the transcript level of a marker gene selected from ISGs, chosen from oligonucleotides,
at least one means for detecting and/or amplifying the DNA or RNA of a respiratory virus.

23. The detection kit as claimed in claim 22, characterized in that it also comprises at least one threshold value stored on a computer-readable medium.

24. The detection kit as claimed in claim 23, characterized in that the threshold value corresponds to the transcript level of said marker gene in a subject who has no infection with a replicating respiratory virus and who preferably has no neutralizing autoantibodies directed toward interferon α and/or has no neutralizing autoantibodies directed toward interferon ω, or in a population of subjects with no infection with a replicating respiratory virus and who preferably do not have neutralizing autoantibodies directed toward interferon α and/or do not have neutralizing autoantibodies directed toward interferon ω.

25. The detection kit as claimed in claim 23, characterized in that it also comprises at least one reference level for said marker gene, stored on a computer-readable medium, said reference level preferably being the transcript level of said marker gene in a subject not infected with a respiratory virus, or in a population of such subjects.

26. The detection kit as claimed in claim 19, characterized in that it also comprises a negative control sample making it possible to ensure the absence of contamination, and/or a positive control sample which corresponds to the level of said marker gene at a concentration representative of the transcript level in a subject with infection with a replicating respiratory virus or in a population of subjects with infection with a replicating respiratory virus.
